(19) United States
(12) Patent Application Publication
(10) Pub. No.: US 2024/0383846 A1
(43) Pub. Date: Nov. 21, 2024

(54) MERCAPTOACETOPHENONE AMINOHYDRAZONES, THEIR SALTS AND USES THEREOF

(71) Applicant: LONDON PHARMACEUTICALS AND RESEARCH CORPORATION, London (CA)

(72) Inventors: ABDEL-RAHMAN LAWENDY, London (CA); MAHMOUD MOHAMED ABDRABO MOUSTAFA, London (CA); ABDELRAHMAN S. MAYHOUB, Giza (EG)

(21) Appl. No.: 18/286,968

(22) PCT Filed: Apr. 14, 2022
(86) PCT No.: PCT/CA2022/050585

§ 371 Date: Oct. 13, 2023

Related U.S. Application Data
(60) Provisional application No. 63/174,833, filed on Apr. 14, 2021.

(51) Int. Cl. C07C 381/00; A61K 9/00; A61K 31/155; A61K 31/407; A61K 31/4164; A61K 31/44; A61K 31/5365; A61K 31/542; A61P 31/04; C07C 317/28; C07D 213/32; C07D 233/24

(52) U.S. Cl. CPC C07C 381/00; A61K 9/0014; A61K 31/155; A61K 31/407; A61K 31/4164; A61K 31/44; A61K 31/5365; A61K 31/542; A61P 31/04; C07C 317/28; C07D 213/32; C07D 233/24

(57) ABSTRACT

A new class of antibiotics, namely, mercaptoacetophenone aminohydrazones, their analogues, their method of preparation, their salts, and their use in cases of bacterial infections, either alone or with other compounds.

### FIELD OF THE INVENTION

The present invention relates to a new class of antibiotics, and in particular to mercaptoacetophenone aminohydrazone antibiotics and their use against bacterial infections. Background

The European Centre for Disease Prevention and Control (ECDC) has reported that healthcare-associated infections (HAIs), together with antimicrobial resistance (AMR), represent one of the most serious health threats not only in Europe but also globally. Nearly 8% of hospitalized European patients experienced adverse events mainly due to nosocomial infections.

Of the bacterial pathogens that are a source of HAIs, methicillin-resistant Staphylococcus aureus (MRSA) remains a persistent problem. MRSA is resistant to not only methicillin but to many antibiotics in our therapeutic arsenal.

Although new derivatives of oxazolidinones and lipoglycopeptides have recently been developed and have increased the therapeutic options available for treatment of MRSA and other Gram-positive multidrug-resistant pathogens, the World Health Organization (WHO) recommended continued development of new therapeutics for those pathogens to keep up with the anticipated evolution of resistance (WHO/EMP/IAU/2017.11).

Apart from HAIs, Staphylococcus aureus is the most predominant organisms responsible for acute diabetic foot infection. Moreover, a recent study indicated the prevalence of MRSA in 10994 diabetic foot infection (DFI) patients was 17%, and 18% among 2147 non-foot skin and soft-tissue infections (Acta Diabetol. 2019; 56(8): 907).

On the other hand, gram-negative bacteria also cause many cases of diabetic foot osteomyelitis (DFO). One study documented 150 cases had a gram-negative isolate (alone or combined with a gram-positive isolate) among 341 cases of DFO, comprising 44.0% of all patients and 50.8% of those with a positive bone culture (Int J Low Extrem Wounds. 2013 March; 12(1):63).

Bacterial strains resistant against common antibiotics have emerged regardless of the mode of action of the drugs. Bacterial species that were once susceptible to several antibiotics have now acquired an array of unique resistance mechanisms. For instance, several strains of Escherichia coli have been found to be insensitive to 3rd and 4th generation carbapenems as well as colistin, the last line of defense against the pathogen. Moreover, some Gram-negative bacteria (G-ve), especially the ESKAPE pathogens: E. coli, Klebsiella pneumoniae, Acinetobacter baumannii, and Pseudomonas aeruginosa have emerged as a serious and growing threat to human health. This is due, in part, to their ability to prevent antibiotic penetration and to over-expression of drug efflux pumps. The problem is mainly exacerbated by the fact that no new chemical scaffold against G-ve pathogens has reached the clinic since the quinolones in the 1960s. Thus, it is clear there is a dire need to develop new antibiotics with novel molecular structures and mechanisms of action.

Several aromatic structures with aminoguanidine side chains were reported with their antibacterial effect. For example, Nishimura, T. et al (YAKUGAKU ZASSHI. 1973 Sep. 25; 93(9):1236-42) reported the synthesis of alkoxybenzaldehyde aminohydrazone derivatives from 4-hydroxybenzaldehyde. The reported derivatives showed weak to moderate antibacterial activity, in which the best derivative demonstrated MIC value of 3.2 ug/mL. In addition, all compounds were tested against laboratory bacterial strains and there was no report for any activity against MDR strains. Lastly, there was no chemical characterization for any compound included in the reference.

U.S. Ser. No. 14/069,089 A1 and many other academic publications (J. Med. Chem. 2014, 57,1609-1615; PloS ONE, 2015, 10, e0130385; Eur. J. Med. Chem. 2015, 94, 306-316; J. Antibiotics. 2015, 68, 259-266; Eur. J. Med. Chem. 2017, 126, 604-613, Eur. J. Med. Chem. 2018, 148, 195-209; Eur. J. Med. Chem. 2019, 175, 49-62) substantially disclosed aminoguanidine-containing compounds connected with phenylthiazole ring system. These references were inclusively limited to a phenylthiazole scaffold.

Aminoguanidine-containing phenylpyrazoles were also reported (J. Med. Chem. 2019,62, 7998-8010) with broad-spectrum antibacterial activity against a larger panel of MRD strains. This reference is also limited to a phenylpyrazole scaffold.

Eur. J. Med. Chem. 2017, 130, 73-85 accounts for several aminoguanidine-containing structures obtained by high-throughput screening. This reference focuses mainly on compounds with a diphenylurea backbone.

In all previous references, the aminoguanidine moiety was an essential element for

antibacterial activity. In addition, a lipophilic side chain was also critical moiety for antibiotic effect.

### SUMMARY OF THE INVENTION

A compound, according to the present invention, has a formula Iₐ, I_{b}, and I_{c}.

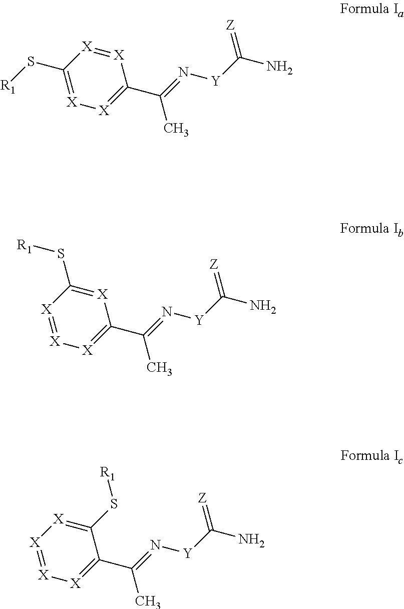

Where R₁ is selected from the group consisting of: a linear or branched akyl chain, unsubstituted or substituted with one or more halides, CN, OH, NO₂, NH, NH₂; an alkenyl, cycloalkyl, alkynyl or cycloakenyl group; and a cycloalkyl or cycloakenyl group connected with a S atom, via a straight or branched one to nine C chain. Each X is independently selected from the group consisting of: C, CH, N, O, S, a halogen, a halogenated C, alkyl, CN, OH, NO₂, NH, NH₂, and a substituted or unsubstituted three to eight C ring. Y is selected from the group consisting of: CH₂, NH, N-alkyl, and a substituted or unsubstituted three to eight C ring. Z is selected from the group consisting of: O, S, NH, N-alkyl, OH, and a substituted or unsubstituted three to eight C ring.

In another embodiment, R₁ is a side group selected from the group consisting of formulas (a)-(o).

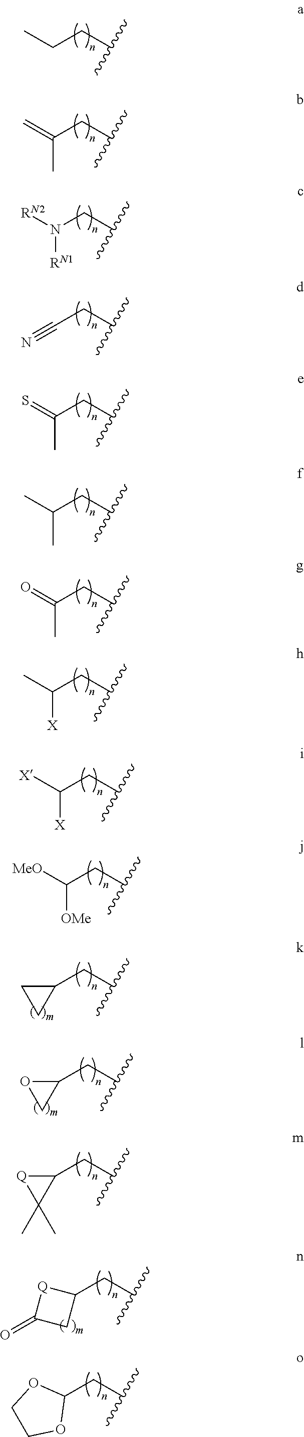

In another embodiment, the compound is a compound of formula 1.

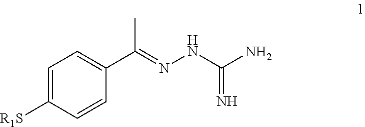

In another embodiment, the compound is a compound of formula 72.

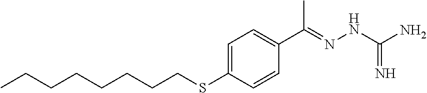 [SMILES: CCCCCCCCSc1ccc(/C(C)=N/NC(=N)N)cc1]

In another embodiment, the compound is a compound of formula 74.

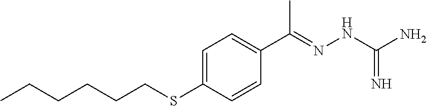 [SMILES: CCCCCCSc1ccc(/C(C)=N/NC(=N)N)cc1]

In another embodiment, the compound is a compound of formula 75.

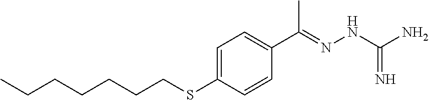 [SMILES: CCCCCCCSc1ccc(/C(C)=N/NC(=N)N)cc1]

In another embodiment, a pharmaceutical composition has a compound, according to the present invention, or a pharmaceutically acceptable salt thereof and one or more pharmaceutically acceptable excipients.

In another embodiment, the pharmaceutical composition is in the form of a topical application.

In another embodiment, a method of treating a patient with a bacterial infection, comprises administering a therapeutically effective amount of a compound, according to the present invention, to a patient in need thereof. In some embodiments, the bacterial infection is a polymicrobial skin infection and the compound is administered in the form of a topical application.

### BRIEF DESCRIPTION OF THE DRAWINGS

In order that the invention may be more clearly understood, a preferred embodiment thereof will now be described in detail by way of example, with reference to the accompanying drawings, in which:

FIG. 1 is a graph showing the change in MIC of a compound, according to the present invention, compared to a control antibiotic.

FIG. 2A is a set of photographs of a skin infection in three test groups, including one treated with a compound, according to the present invention.

FIG. 2B is a box plot of bacterial counts recovered from the skin lesions of the test groups illustrated in FIG. 2A.

### DESCRIPTION OF THE INVENTION

In the present invention, chemical entities useful for antibacterial activity are linked to a mercaptophenyl ring in order to increase the atomic efficiency, compared to related antibacterial compounds. The new scaffold, according to the present invention, has the advantage of small molecular weight, drug-like properties, and good aqueous solubility.

Compounds according to the present invention possess an additional advantage over the reported compounds (e.g., YAKUGAKU ZASSHI. 1973 Sep. 25; 93(9):1236-42), due to the presence of a methyl group on the benzylic carbon. This methyl adds further protection to the C═N bond (imine bond). Consequently, the new scaffold has high chemical and enzymatic stability. Other advantages include the availability of shorter, efficient, and economic synthetic protocols.

In some illustrative embodiments, the present invention comprises compounds having formula I_{a-c}, or pharmaceutically acceptable salts, hydrates, or solvates thereof.

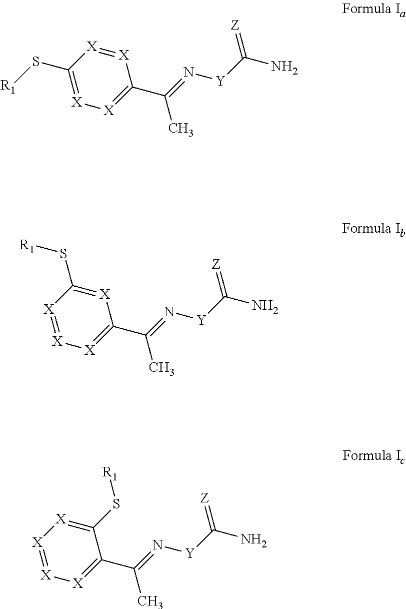

In some illustrative embodiments, the present invention comprises compounds having formula II_{a-c}, or pharmaceutically acceptable salts, hydrates, or solvates thereof.

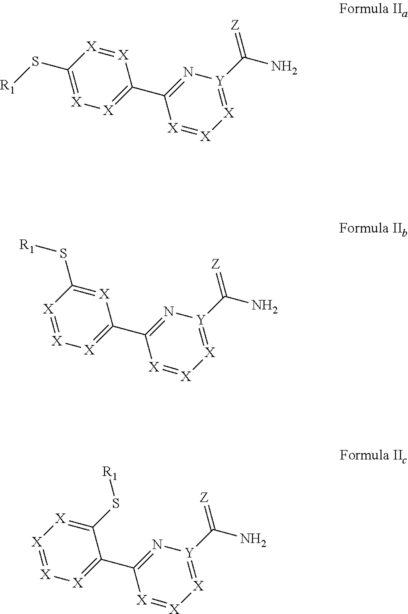

In some illustrative embodiments, the present invention comprises compounds having formula III_{a-c}, or pharmaceutically acceptable salts, hydrates, or solvates thereof.

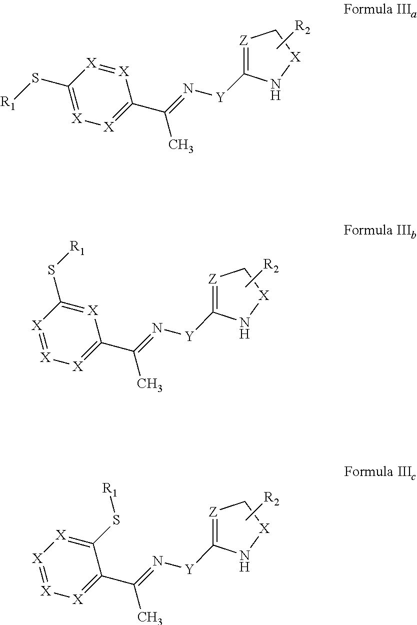

In some other embodiments, the present invention comprises a pharmaceutical composition comprising a compound disclosed herein, or a pharmaceutically acceptable salt thereof, together with one or more pharmaceutically acceptable diluents, and excipients.

In some embodiments, the present invention comprises a pharmaceutical composition comprising a compound disclosed herein, in combination with one or more other therapeutically active compounds by the same or different mode of action, and one or more pharmaceutically acceptable excipients.

In some embodiments, the present invention comprises a method for treating a patient of bacterial infection, the method comprising the step of administering a therapeutically effective amount of a compound disclosed herein, together with one or more pharmaceutically acceptable excipients, to the patient in need of relief from said bacterial infection. The bacterial infection may be topical or systemic. Preferably, the bacterial infection is topical.

In some embodiments, the present invention comprises a method for treating a patient of bacterial infection, the method comprising the step of administering a therapeutically effective amount of a compound disclosed herein, in combination with one or more therapeutically effective compounds by the same or different mode of action, together with one or more pharmaceutically acceptable excipients, to the patient in need of relief from said bacterial infection.

In some other embodiments, the present invention comprises a method for treating a patient of bacterial infection, the method comprising the step of administering a therapeutically effective amount of a compound of formulas I-IV, to the patient in need of relief from said bacterial infection.

In some illustrative embodiments, R₁ is a linear or branched alkyl carbon chain. R₁ may be substituted with a halide, CN, OH, NO₂, NH, NH₂ or any related substituents. The substitution of R₁ may be mono-, di- or multi substitution(s). R₁ may be a saturated or unsaturated carbon chain including, but not limited to an alkenyl, cycloalkyl, alkynyl or cycloalkenyl moiety. The R₁ side chain may contain ketonic, aldehydic or epoxy functionalities. R₁ may also be a cyclic structure, cycloalkyl or cycloalkenyl, connected with a S atom, via a carbon chain that includes a number of carbon atoms from C₁ to C₉, such as methylene, ethylene, isopropylene, straight or branched and the like.

In some illustrative embodiments, R₂ is a small alkyl substituent that includes a number of carbon atoms from C₁ to C₄. Preferably, R₂ is a methyl or an ethyl group. R₂ may also be a halide, CN, OH, NO₂, NH, NH₂ or any related substituents. The substitution of R₂ may be mono-, di- or multi substitution(s).

In another embodiment, compounds of the present invention include their respective isomers (stereo or structural) either as bases, salts, or mixtures thereof.

In some illustrative embodiments, each X group is independently CH, N, O or S. Where X is a C, it may be halo substituted. It may be also substituted with one or more halogens (e.g. F), halogenated alkyl (e.g. CF₃), alkyl, CN, OH, NO₂, NH, NH₂ or any related substituents. X may be a part of three, four, five, six, seven, or eight membered rings. Preferably, X is a part of a five or a six membered ring.

In some illustrative embodiments, Y is independently CH₂ or NH, optionally substituted with an alkyl group, preferably a methyl group. Y may also be a part of three, four, five, six, seven, or eight membered rings. Preferably, Y is a part of a five or a six membered ring. Y may be a part of cyclic, heterocyclic, aromatic, or heteroaromatic rings, preferably, substituted or unsubstituted imidazoline, imidazole, triazole, pyridine, or pyrimidine.

In some illustrative embodiments, Z is independently O, S, NH, optionally substituted with an alkyl group, preferably a methyl group, or OH. Z may also be a part of three, four, five, six, seven, or eight membered rings. Preferably, Z is a part of a five or a six membered ring. Optionally, Z can be a part of cyclic, heterocyclic, aromatic, or heteroaromatic rings, preferably, substituted or unsubstituted imidazoline, imidazole, or triazole.

Certain preferred embodiments of the present invention include the following examples of formulas 1 to 45:

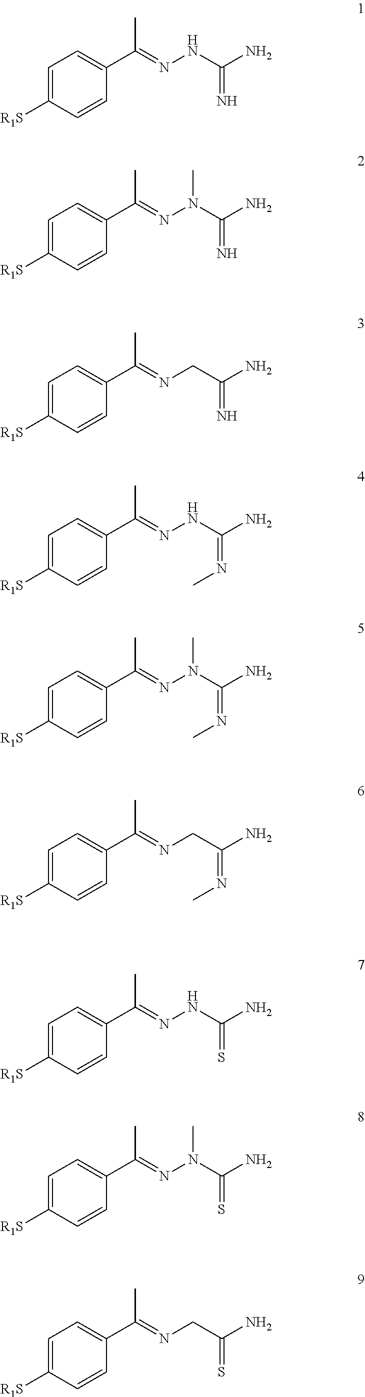

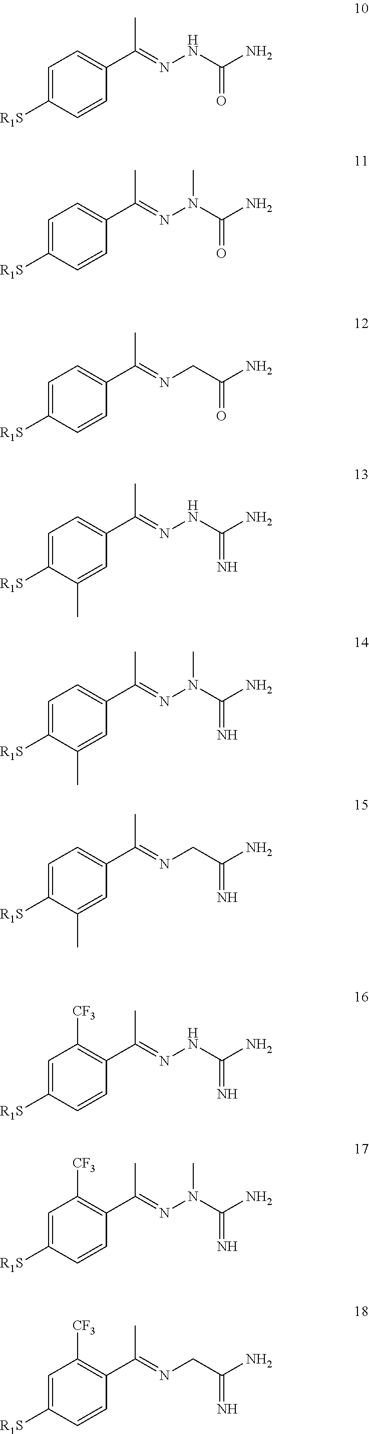

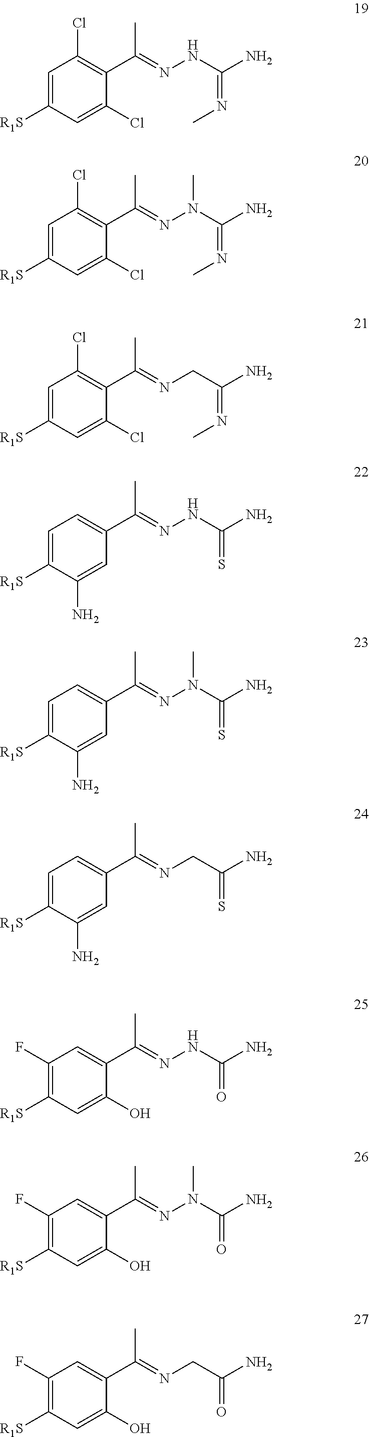

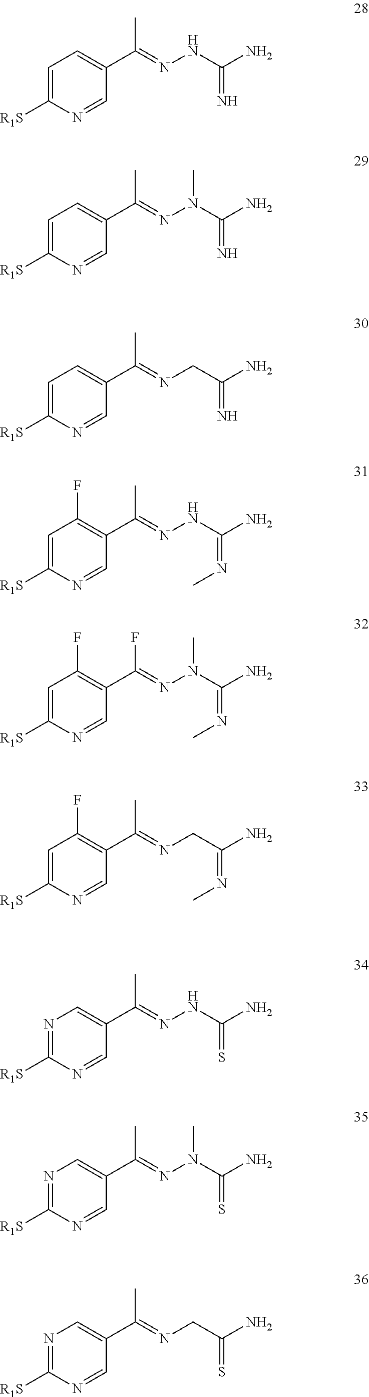

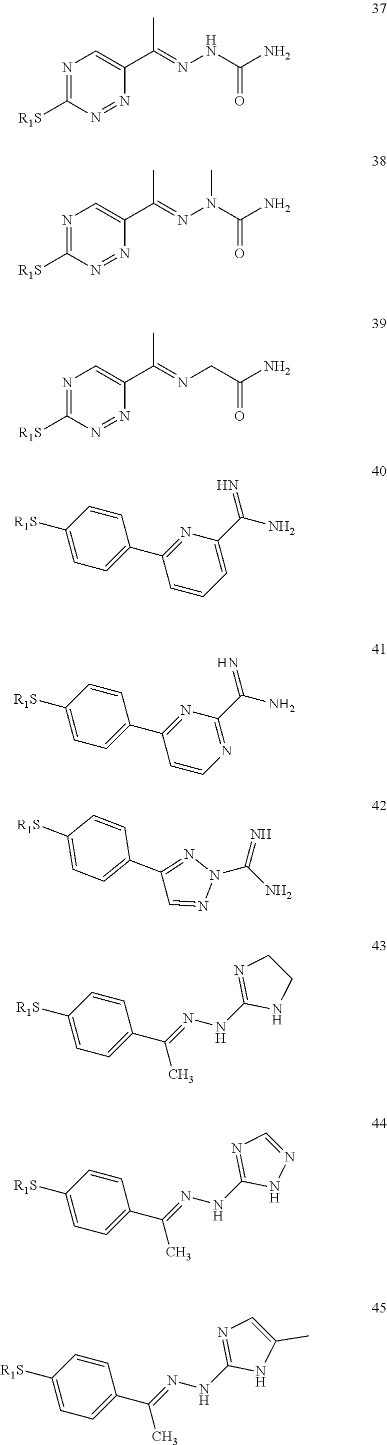

In the above example formulas 1-45, R₁ is selected from the group consisting of a-o:

| entry | R1 |
|---|---|
| a | 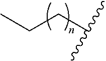 |
| b | 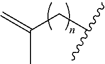 |
| c | 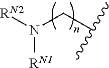 |
| d | 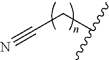 |
| e | 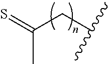 |
| f | 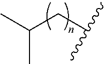 |
| g | 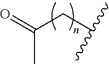 |
| h | 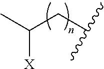 |
| i | 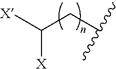 |
| j | 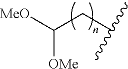 |
| k | 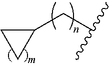 |
| l | 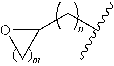 |
| m | 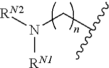 |
| n | 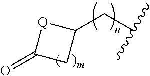 |
| o | 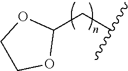 |

The value of n ranges from 0 to 12, while the value of m ranges from 0 to 7. The group represented by Q may be NH, O, S, or N—R^{N1}. The group represented by R^{N1} and R^{N2} are, independently, alkyl or cycloalkyl groups with between 1 and 7 C atoms and may be optionally substituted with one or more halogen atoms. The groups represented by X and X′ are, independently selected from F, Cl, Br, or I.

In some other embodiments, additional to the thioether, S atom may be in any oxidation state, preferably, sulfoxide or sulfone, as shown in Formulas IV_{a-f}.

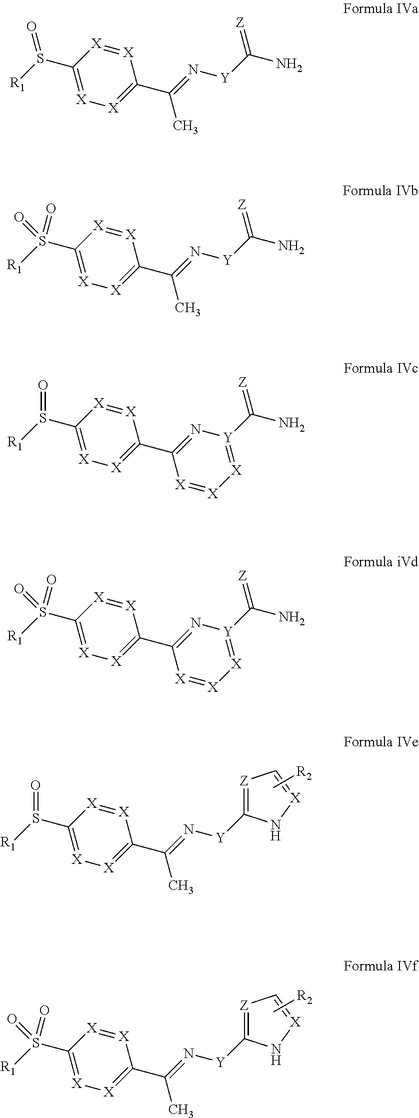

Certain preferred embodiments of the present invention include the following examples of formulas 46-60:

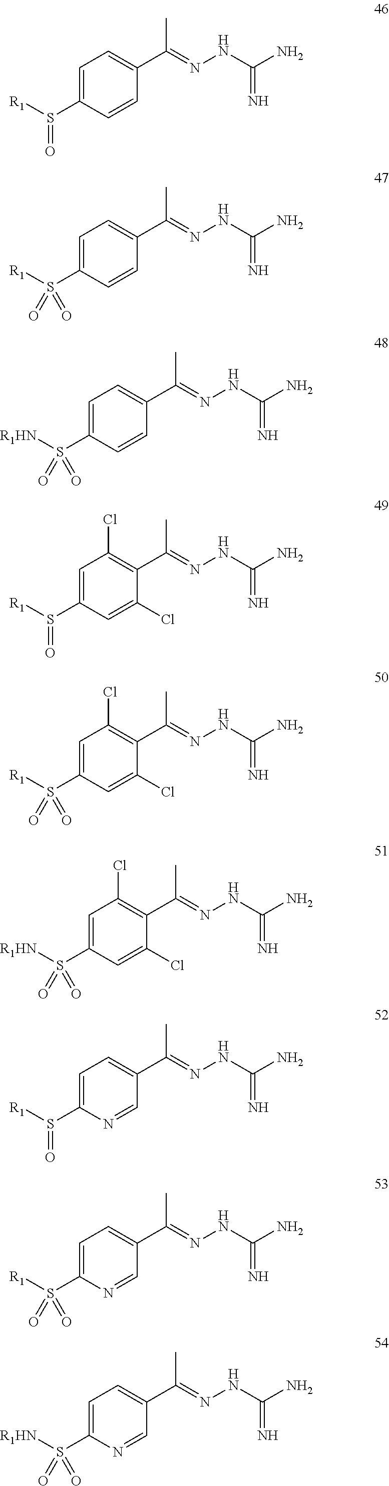

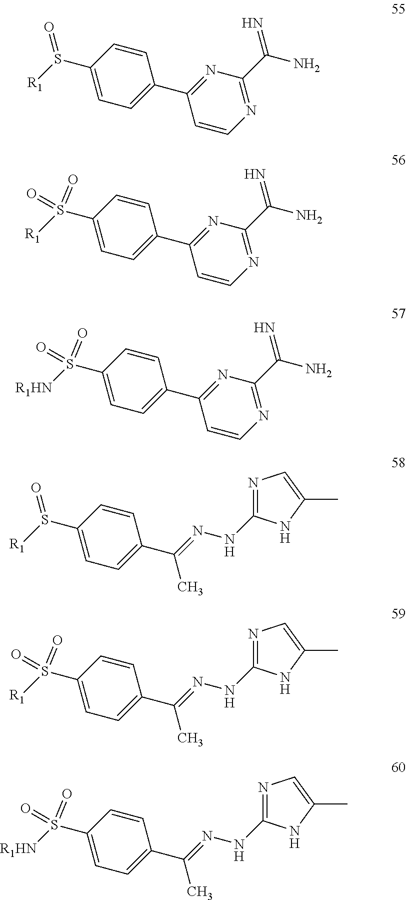

In the above example formulas 46-60, R₁ is selected from the group consisting of a-o:

| entry | R1 |
|---|---|
| a | 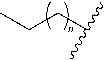 |
| b | 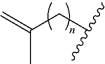 |
| c | 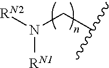 |
| d | 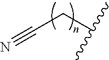 |
| e | 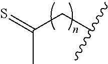 |
| f | 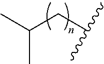 |
| g | 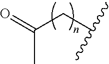 |
| h | 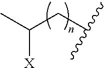 |
| i | 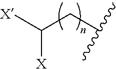 |
| j | 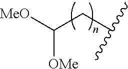 |
| k | 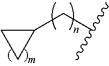 |
| l | 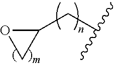 |
| m | 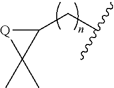 |
| n | 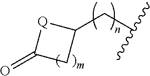 |
| o | 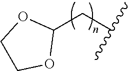 |

The value of n ranges from 0 to 12, while the value of m ranges from 0 to 7. The group represented by Q may be NH, O, S, or N—R^{N1}. The group represented by R^{N1} and R^{N2} are, independently, alkyl or cycloalkyl groups with between 1 and 7 C atoms and may be optionally substituted with one or more halogen atoms. The groups represented by X and X′ are, independently selected from F, Cl, Br, or I.

In some illustrative embodiments, the present invention includes salts between compounds belonging to Formulas I to IV and any acidic counterpart. Preferably, the acidic counterpart is one or more acidic antimicrobial compounds such as penicillins, penams, carbapenams, clavams, cephems, carbacephems, oxacephems, monobactam, quinolones and fluoroquinolones.

Certain preferred embodiments of the present invention include the following examples of formulas 61-65:

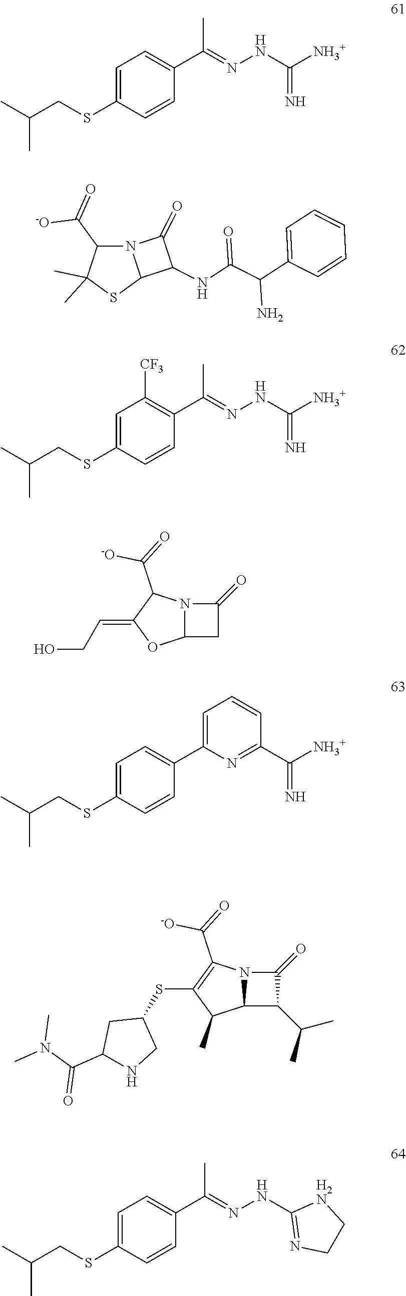

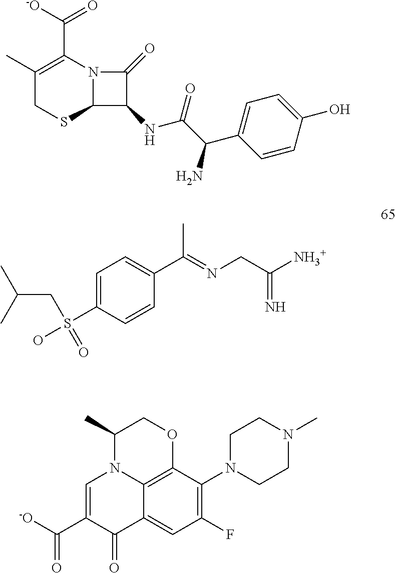

In some illustrative embodiments, the present invention includes salts between compounds belonging to Formulas I to IV and any two or more acidic counterparts. One of the acidic counterparts may be β-lactamase inhibitor, and the other one may be an antimicrobial agent with acidic function group.

Certain preferred embodiments of the present invention include the following examples of formulas 66 and 67:

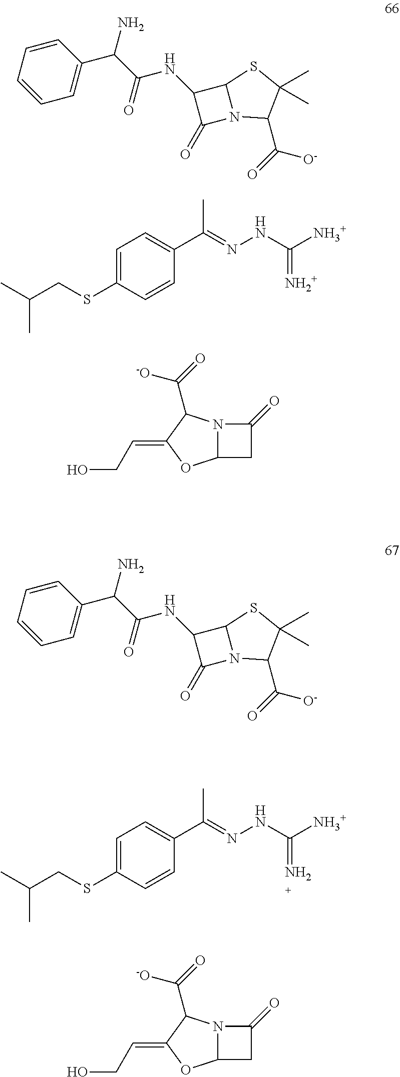

The compounds described herein may be used alone or in combination with other antimicrobials that may be therapeutically effective by the same or different modes of action. In addition, the compounds described herein may be used in combination with other therapeutics that are administered to treat other symptoms of bacterial infections, such as compounds administered to relieve pain, allergy, swelling, nausea/vomiting, and the like.

As used herein, the following terms and phrases shall have the meanings set forth below. Unless defined otherwise, all technical and scientific terms used herein have the same meaning as commonly understood to one of ordinary skill in the art.

In the present disclosure the term “about” can allow for a degree of variability in a value or range, for example, within 1%, within 5%, or within 10% of a stated value or of a stated limit of a range. In the present disclosure the term “substantially” can allow for a degree of variability in a value or range, for example, within 90%, within 95%, 99%, 99.5%, 99.9%, 99.99%, or at least about 99.999% or more of a stated value or of a stated limit of a range.

A “halogen” designates F, Cl, Br or I. A “halogen substitution” or “halo” substitution designates replacement of one or more hydrogen atoms with F, Cl, Br or I.

As used herein, the term “alkyl” refers to a saturated monovalent chain of carbon atoms, which may be optionally branched. It is understood that in embodiments that include alkyl, illustrative variations of those embodiments include lower alkyl, such as C₁ to C₉ alkyl, methyl, ethyl, propyl, 3-methylbutyl, and the like.

As used herein, the term “alkenyl” refers to an unsaturated monovalent chain of carbon atoms including at least one double bond, which may be optionally branched. It is understood that in embodiments that include alkenyl, illustrative variations of those embodiments include lower alkenyl, such as C₂-C₆ alkenyl, and the like.

As used herein, the term “alkynyl” refers to an unsaturated monovalent chain of carbon atoms including at least one triple bond, which may be optionally branched. It is understood that in embodiments that include alkynyl, illustrative variations of those embodiments include lower alkynyl, such as C₂-C₆ alkynyl, and the like.

As used herein, the terms “cycloalkyl” refers to a monovalent chain of carbon atoms, a portion of which forms a ring. It is understood that in embodiments that include cycloalkyl, illustrative variations of those embodiments include lower cylcoalkyl, such as C₃-C₆ cycloalkyl, cyclopropyl, cyclobutyl, 3-methylcyclohexyl, and the like.

As used herein, the term “cycloalkenyl” refers to an unsaturated monovalent chain of carbon atoms, a portion of which forms a ring. It is understood that in embodiments that include cycloalkenyl, illustrative variations of those embodiments include lower cycloalkenyl, such as C₃-C₆ cycloalkenyl, cyclopentenyl, cyclohexenyl, and the like.

It is understood that each of alkyl, cycloalkyl, alkenyl, and cycloalkenyl may be optionally substituted with independently selected groups such as halide, alkyl, halogenated alkyl, alkoxy, hydroxy, hydroxyalkyl, carboxylic acid and derivatives thereof, including esters, nitrile, amides, and nitrites, acyloxy, aminoalkyl and dialkylamino, acylamino, thio, and the like, and combinations thereof.

The term “optionally substituted,” or “optional substituents,” as used herein, means that the groups in question are either unsubstituted or substituted with one or more of the substituents specified. When the groups in question are substituted with more than one substituent, the substituents may be the same or different. Moreover, when using the terms “independently,” means that the groups in question may be the same or different. Certain of the herein defined terms may occur more than once in the structure, and upon such occurrence each term shall be defined independently of the other.

The term “patient” includes human and non-human animals such as companion animals, including horses, dogs, cats and the like, and livestock animals. Livestock animals are animals raised for production of food, textiles, or other animal-based products. The patient to be treated is preferably a mammal and, more preferably, a human.

The term “pharmaceutically acceptable diluent” or “pharmaceutically acceptable excipient” are art-recognized and refer to a pharmaceutically acceptable material, composition or vehicle, such as a liquid or solid filler, solvent or encapsulating material, involved in carrying or transporting any subject composition or component thereof. Each carrier must be “acceptable” in the sense of being compatible with the subject composition and its components and not injurious to the patient. Some examples of materials which may serve as pharmaceutically acceptable carriers include: sugars, such as lactose and maltose; starches, such as corn starch and gelatinized starch; cellulose, and its derivatives, such as carboxymethyl cellulose salt, and hydroxypropylmethyl cellulose; thickening agents such as gelatin and tragacanth; disintegrants such as copovidone; other excipients, such as cocoa butter and suppository waxes and pyrogen-free water for sterile products; and other non-toxic compatible substances employed in pharmaceutical formulations.

As used herein, the term “administering” includes all means of introducing the compounds and compositions described herein to the patient, including, but are not limited to, topical, oral, intravenous, intramuscular, transdermal, inhalation, buccal, ocular, vaginal, rectal, and the like. The compounds and compositions described herein may be administered in unit dosage forms or formulations containing conventional nontoxic pharmaceutically acceptable carriers, adjuvants, and vehicles.

To obtain the compounds described herein, Schemes 1-4 were adopted. According to Scheme 1,4-mercaptoacetophenone, obtained from the corresponding 4-bromoacetophenone, was dissolved in acetone and allowed to react with alkyl bromide in the presence of potassium carbonate as a base. The S-alkyl product was then charged with 1.2 equivalent aminoguanidine hydrochloride in acetic acid to furnish the desired finished products as yellow solids.

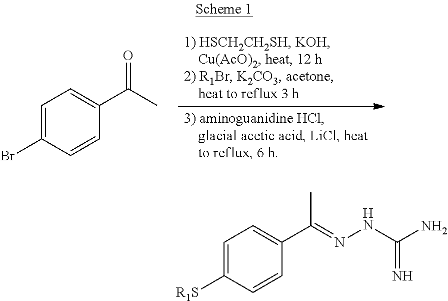

An alternative pathway to obtain the compounds described herein is illustrated in Scheme 2, in which the “RS” side chain can be tethered to the aromatic ring via metal-catalyzed chemistry. In brief, 4-bromoacetophenenone (1 equiv.) is dissolved in a degassed and dry dioxane (10 mL). Xantphos, as a ligand, DIPEA, as a base, and Pd (dba) as a catalyst were added. The reaction mixture was kept under argon atmosphere and heat at reflux temperature for 5-20 h. After reaction completion as detected by TLC, the reaction mixture was passed through Celite 545, washed with copious amount of EtOAc, and concentrated under reduced pressure. The obtained organic material was then purified by normal-phase column chromatography using Hexane:EtOAc (9:1). Finally, the aminoguanidine was added, as described in Scheme 1.

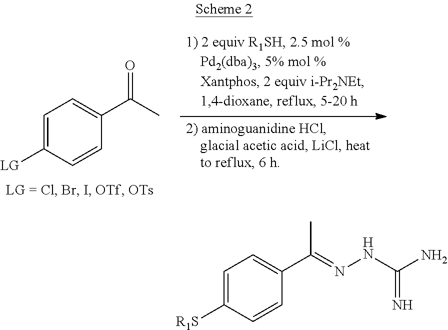

In some illustrative embodiment, “LG” may be any halide, triflate, mesylate, or any other leaving group used in metal-catalyzed chemistry.

In some illustrative embodiment, Xantphos can be replaced with other phosphine or non-phosphine ligands, including triphenyl phosphine, tricyclopentyl phosphine, tricyclohexyl phosphine, XPhos, tButXPhos, SPhos, sSPhos, DavePhos, t-Bu-Xantphos, any ferrocene derivative carbene such limited ligands, as but not to, DPPF, [1,1′-Bis(diphenylphosphino)ferrocene]tetracarbonylchromium(0), 1,1′-Bis(diisopropylphosphino)ferrocene, 1,1′-Bis(di-tert-butylphosphino)ferrocene, 1,3-Bis (2,4,6-trimethylphenyl)imidazolinium chloride, 1,3-Bis(2,6-diisopropylphenyl)-1,3-dihydro-2H-imidazol-2-ylidene, 1,3-Bis(2,4,6-trimethylphenyl)-4,5-dihydroimidazol-2-ylidene, and Chloro1,3-Bis(2,4,6-trimethylphenyl)imidazol-2-ylidenecopper(I).

In some illustrative embodiment, Bis(dibenzylideneacetone)palladium(0), can be replaced with other palladium, copper or nickel catalyst(s), including Pd acetate, XPhos Pd G1, XPhos Pd G2, XPhos Pd G3, QPhos Pd G1, QPhos Pd G2, QPhos Pd G3, Tris(dibenzylideneacetone)dipalladium(0), Tetrakis(triphenylphosphine)palladium(0), (Ethylenediamine)palladium(II) chloride, 1,4-Bis(diphenylphosphino)butane-palladium(II) chloride, Bis(benzonitrile)palladium(II) chloride, (1,3-Bis(diphenylphosphino)propane)palladium(II) chloride, [(+)-2,2′-Bis(di-p-tolylphosphino)-1,1′-binaphtyl]palladium(II) chloride, [(+)-2,2′-Bis(diphenylphosphino)-1,1′-binaphthyl]palladium(II) chloride, 2-(Dimethylaminomethyl)ferrocen-1-yl-palladium(II) chloride, and 1,1′-Bis(diisopropylphosphino)ferrocene.

Other compounds described herein may be prepared according to Scheme 3, in which 4-acetylbenzenesulfonyl chloride (1 equiv.) in dry DCM (10 mL) was treated with appropriate amine in a slight excess (1.2 equiv.) and triethyl amine (2 equiv.). The reaction mixture was stirred at ambient temperature for 5-10 h. After reaction completion as detected by TLC, the reaction mixture was concentrated under reduced pressure. The obtained organic material was then purified by normal-phase column chromatography using Hexane:EtOAc (1:1). Finally, the aminoguanidine was added as described in Scheme 1.

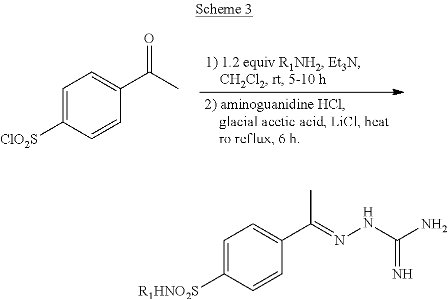

Certain sulfone and sulfoxide compounds described herein may be prepared according to Scheme 4. In brief, the substituted mercaptoacetophenone can be oxidized using any oxidizing agent, such as, but not limited to m-chloroperbenzoic acid (mCPBA). The sulfone and sulfoxide derivatives were obtained in high yield by controlling the molar ratio and reaction temperature as illustrated in Scheme 4. Finally, the aminoguanidine was added as described in Scheme 1.

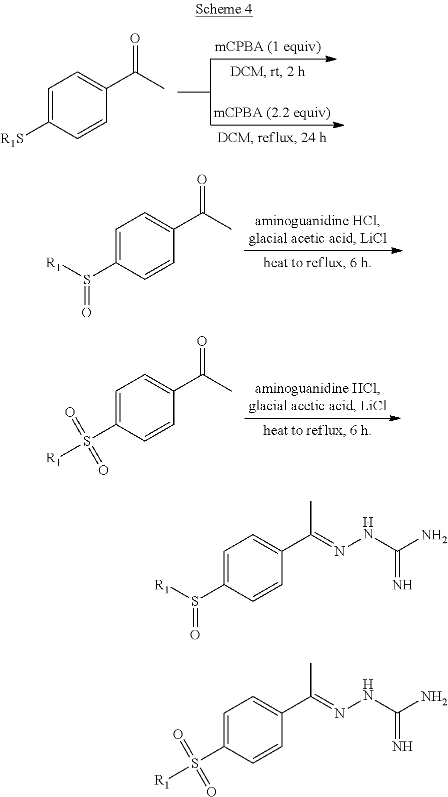

Table 1 summarizes the antibacterial activity of certain compounds, according to the present invention, as calculated by minimum inhibitory concentration (MIC) values against one multidrug resistant gram-positive strain (MRSA USA300), and one multidrug resistant gram-negative (Acinetobacter baumannii AB5075). The later bacterial strain is listed at the top of the WHO's critical priority list for antibiotic development. Preferred compounds, according to the present invention are example compounds of formulas 72, 74, and 75, and, in particular, example compound of formula 75.

TABLE 1
Determination of the minimum inhibitory concentration (MIC) of the new compounds and
reference drugs against MRSA USA300 and A. baumannii AB5075

| | | MIC | | | |
|---|---|---|---|---|---|
| Example Compound | | MRSA | | | |
| Formula | | USA300 MIC | | A. baumannii AB5075 MIC | |
| 68. | 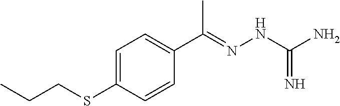 [SMILES: CCCSc1ccc(/C(C)=N/NC(=N)N)cc1] | No inhibition | | No inhibition | |
| 69. | 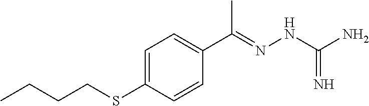 [SMILES: CCCCSc1ccc(/C(C)=N/NC(=N)N)cc1] | 8 | ug/mL | 16 | ug/mL |
| 70. | 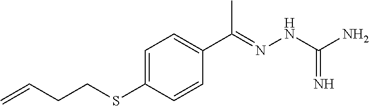 [SMILES: C=CCCSc1ccc(/C(C)=N/NC(=N)N)cc1] | 32 | ug/mL | 32 | ug/mL |
| 71. | 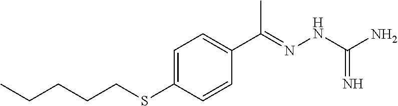 [SMILES: CCCCCSc1ccc(/C(C)=N/NC(=N)N)cc1] | 8 | ug/mL | 16 | ug/mL |
| 72. | 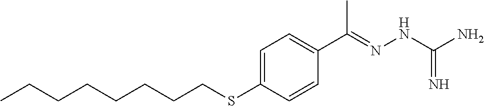 [SMILES: CCCCCCCCSc1ccc(/C(C)=N/NC(=N)N)cc1] | 1 | ug/mL | 8 | ug/mL |
| 73. | 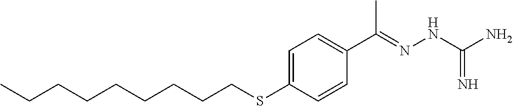 [SMILES: CCCCCCCCCSc1ccc(/C(C)=N/NC(=N)N)cc1] | 2 | ug/mL | 16 | ug/mL |
| 74. | 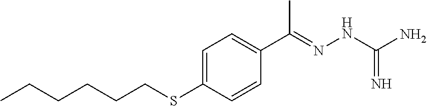 [SMILES: CCCCCCSc1ccc(/C(C)=N/NC(=N)N)cc1] | 0.5 | ug/mL | 4 | ug/mL |
| 75. | 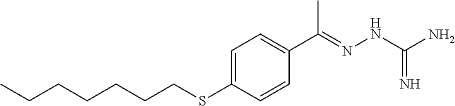 [SMILES: CCCCCCCSc1ccc(/C(C)=N/NC(=N)N)cc1] | 0.5 | ug/mL | 1 | ug/mL |
| 76. | 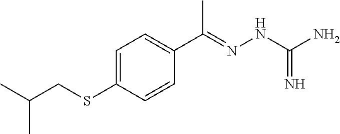 [SMILES: C/C(=N\NC(=N)N)c1ccc(SCC(C)C)cc1] | 8 | ug/mL | 16 | ug/mL |
| 77. | 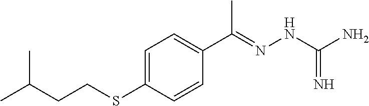 [SMILES: C/C(=N\NC(=N)N)c1ccc(SCCC(C)C)cc1] | 4 | ug/mL | 8 | ug/mL |
| 78. | 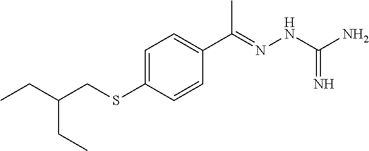 [SMILES: CCC(CC)CSc1ccc(/C(C)=N/NC(=N)N)cc1] | No inhibition | | No inhibition | |
| 79. | 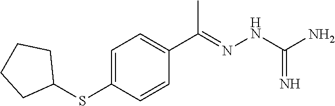 [SMILES: C/C(=N\NC(=N)N)c1ccc(SC2CCCC2)cc1] | 8 | ug/mL | 16 | ug/mL |
| 80. | 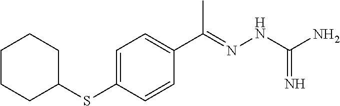 [SMILES: C/C(=N\NC(=N)N)c1ccc(SC2CCCCC2)cc1] | 4 | ug/mL | 8 | ug/mL |
| 81. | 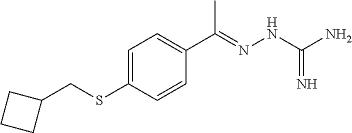 [SMILES: C/C(=N\NC(=N)N)c1ccc(SCC2CCC2)cc1] | 8 | ug/mL | 16 | ug/mL |
| 82. | 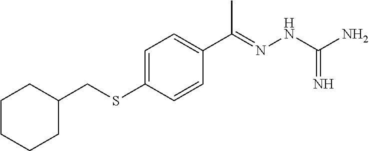 [SMILES: C/C(=N\NC(=N)N)c1ccc(SCC2CCCCC2)cc1] | No inhibition | | No inhibition | |
| 83. | 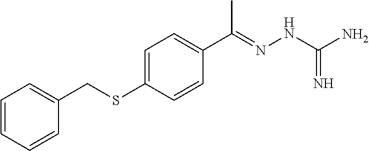 [SMILES: C/C(=N\NC(=N)N)c1ccc(SCc2ccccc2)cc1] | 4 | ug/mL | 16 | ug/mL |
| 84 | 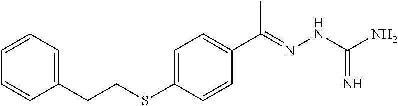 [SMILES: C/C(=N\NC(=N)N)c1ccc(SCCc2ccccc2)cc1] | 2 | ug/mL | 8 | ug/mL |

| | | | | |
|---|---|---|---|---|
| Vancomycin | 1.0 | ug/mL | >128 | ug/mL |

| | | |
|---|---|---|
| Gentamycin | Not tested | 1 |

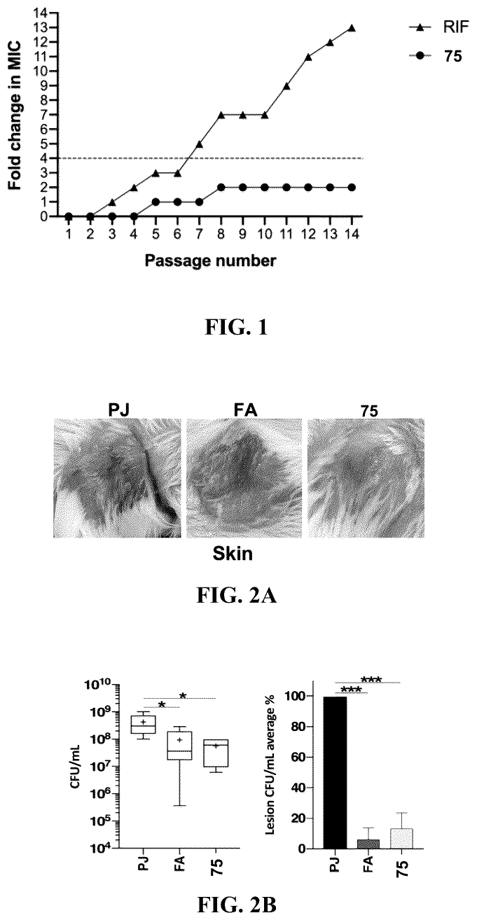

A multi-step resistance experiment was conducted, with example compound of formula 75. Initially, the MICs of heptyl compound and rifampicin, as the control antibiotic, were determined against MRSA USA300 using the broth microdilution method. Then, the strain was subcultured for fourteen consecutive passages over two weeks, with increasing concentrations of the tested agents, to detect the shift in the MIC, if any. As shown in FIG. 1, the MIC value for heptyl compound increased one-fold on the fifth passage, then 2-fold on the eighth passage and remained stable till the end of the experiment. On the other hand, MRSA USA300 developed rapid resistance to the rifampicin antibiotic, with MIC increasing 7-fold after eight passages and continued to increase till it reached 13-fold increase by the end of the experiment. These results indicate that USA300 is less likely to develop rapid resistance to certain compounds according to the present invention, compared with the control antibiotic (rifampicin).

The effectiveness of certain compounds, according to the present invention, was assessed using a murine skin infection model, conducted as previously described (Tseng, C. W. et. al. Subcutaneous infection of methicillin resistant Staphylococcus aureus (MRSA), J. Vis. Exp., (2011) 2528). The effect of example compound of formula 75 was assessed against skin infection caused by methicillin-resistant Staphylococcus aureus (MRSA), the main causative agent of diabetic foot ulcer. Within 72 hours post-subcutaneous infection with MRSA strain USA300, the backs of the mice started showing evident lesions. At this point, treatment regimen started for the three groups of mice, for four days, twice daily, with petroleum jelly (PJ) or petroleum jelly with 2% example compound of formula 75 or 2% fusidic acid ointment (FA). Visually comparing the skin lesions of the three mice groups showed a significant discrepancy between them. The mice with plain vehicle (PJ) applied throughout the experiment had ulcerated lesions and visible skin damage with demarcated red prominent edges, as shown in FIG. 2A. On the other hand, both example compound of formula 75, and fusidic acid (FA)-treated mice showed signs of lesion healing and better recovery during the infection model, as shown in FIG. 2A. By the end of the experiment, the bacterial counts recovered from the skin lesions were compared and it was observed that example compound of formula 75 caused a comparable decrease (˜1 log cycle or 90% reduction) in the bacterial loads to that of the fusidic acid, as shown in FIG. 2B, both significantly lower than the counts obtained from the PJ group (*p≤0.05).

Without wishing to be bound by theory, the structure-activity relationships (SAR) suggest that these compounds are target specific since a small change in structure is accompanied by a significant change in antibacterial effect. In brief, the optimum antimicrobial activity was observed with the heptyl side chain (i.e. example compound of formula 75) in which it showed a low MIC value against both tested microorganisms, S. aureus USA300 and A. baumannii AB5075. The hexyl derivative (example compound of formula 74) maintained the same potency against S. aureus USA300, but it was four times less potent against the tested gram-negative pathogen (see Table 1, above).

The effectiveness of certain preferred compounds, according to the present invention, against both Gram-positive and Gram-negative bacterial strains is particularly useful in treating polymicrobial infections. For example, certain compounds, according to the present invention, may be particularly useful in the treatment of polymicrobial skin infections such as cellulitis, diabetic foot infections, skin abscesses, or necrotizing skin infections, due to the effectiveness of such compounds against both Gram-positive and Gram-negative bacteria.

### Preparation of Example Compounds

Certain preferred compounds, according to the preset invention, may be prepared according to the following method. First, 4-mercaptoacetophenone is prepared from its 4-bromo analogue. This may be done by dissolving 4-bromoacetophenone (200 mg, 1 mmol) in dry DMSO (10 mL) and charging the reaction mixture with ethane-1,2-dithiol (1.5 equiv.) and copper II acetate (25 mg), and heating at 140° C. for 12 hours. After reaction completion, as confirmed by TLC, the reaction mixture is quenched by distilled water (100 mL), the solid material is collected and purified by crystallization from ethanol 70%. The obtained 4-mercaproacetophenone (150mg, 1 mmol) is dissolved in acetone and allowed to react with alkyl bromide (2 equiv.) in the presence of potassium carbonate (200 mg) as a base. After completion of reaction, the reaction mixture is concentrated under reduced pressure and the product is purified by column chromatography using silica gel as a solid phase and ethyl acetate:hexane (1:9) as a mobile phase. The S-alkyl product is then charged with 1.2 equivalent aminoguanidine hydrochloride in acetic acid to furnish the desired finished products as solids. The following products are examples of products characterized from this method.

2-(1-(4-(Propyllthio)phenyl)ethylidene)hydrazine-1-carboximidamide. Beige powder (170 mg, 66%). ¹H NMR (DMSO-d₆) δ: 7.76 (d, J=8.1 Hz, 2H), 7.28 (d, J=8.1 Hz, 2H), 5.86 (brs, 2H), 5.47 (brs, 2H), 2.97 (t, J=8.4 Hz, 2H), 2.22 (s, 3H), 1.66-1.54 (m, 2H), 1.01(t, J=8.4 Hz, 3H); ¹³C NMR (DMSO-d₆); δ 160.11, 147.15, 138.06, 135.50, 128.14, 126.33, 34.57, 22.98, 22.44, 13.64; MS (m/z); 250.36 (M⁺, 100%).

2-(1-(4-(Butylthio)phenyl)ethylidene)hydrazine-1-carboximidamide. Light brown powder (190 mg, 75%). ¹H NMR (DMSO-d₆) δ: 7.75 (d, J=8.1 Hz, 2H), 7.26 (d, J=8.1 Hz, 2H), 5.84 (brs, 2H), 5.45 (brs, 2H), 2.98 (t, J=8.2 Hz, 2H), 2.20 (s, 3H), 1.60-1.52 (m, 2H), 1.44-1.38 (m, 2H), 0.90 (t, J=8.2 Hz, 3H); ¹³C NMR (DMSO-d₆); δ 160.12, 147.14, 138.05, 135.55, 128.08, 126.32, 32.26, 31.20, 21.75, 13.97, 13.63; MS (m/z); 264.36 (M⁺, 100%).

2-(1-(4-(But-3-en-1-ylthio)phenyl)ethylidene)hydrazine-1-carboximidamide. Brown powder (180 mg, 71%). ¹H NMR (DMSO-d₆) δ: 7.76 (d, J=8.1 Hz, 2H), 7.28 (d, J=8.1 Hz, 2H), 5.94 (ddt, J=16.4, 10.2, 7.4 Hz, 1H), 5.55 (brs, 4H), 5.12 (dd, J=10.2, 2.5 Hz, 2H), 3.05 (t, J=7.3 Hz, 2H), 2.77-2.61 (m. 2H), 2.36 (s, 3H); ¹³C NMR (DMSO-d₆); δ 138.11, 137.47, 137.04, 135.29, 128.30, 126.40, 116.79, 116.43, 33.26, 32.00, 13.65; MS (m/z); 262.37 (M⁺, 100%).

2-(1-(4-(Pentylthio)phenyl)ethylidene)hydrazine-1-carboximidamide 6. White powder (205 mg, 82%). ¹H NMR (DMSO-d₆) δ: 7.76 (d, J=8.2 Hz, 2H), 7.26 (d, J=8.2 Hz, 2H), 5.89 (brs, 2H), 5.51 (brs, 2H), 2.97 (t, J=7.2 Hz, 2H), 2.20 (s, 3H), 1.61-1.54 (m, 2H), 1.41-1.25 (m, 4H), 0.88 (t, J=7.2 Hz, 3H); ¹³C NMR (DMSO-d₆); δ 160.10, 147.16, 138.00, 135.59, 128.06, 126.44, 32.53, 31.05, 28.76, 22.19, 14.32, 13.64; MS (m/z); 278.41 (M⁺, 100%).

2-(1-(4-(Heptylthio)phenyl)ethylidene)hydrazine-1-carboximidamide. Light brown powder (210 mg, 86%). ¹H NMR (DMSO-d₆) δ: 7.74 (d, J=8.1 Hz, 2H), 7.25 (d, J=8.1 Hz, 2H), 5.85 (brs, 2H), 5.47 (brs, 2H), 2.96 (t, J=7.1 Hz, 2H), 2.21 (s, 3H), 1.61-1.53 (m, 2H), 1.42-1.20 (m, 8H), 0.87 (t, J=7.1 Hz, 3H); ¹³C NMR (DMSO-d₆); δ 160.14, 147.11, 138.06, 135.56, 128.09, 126.31, 32.60, 31.63, 29.08, 28.69, 28.54, 22.49, 14.39, 13.64; MS (m/z); 306.47 (M⁺, 100%).

2-(1-(4-(Octylthio)phenyl)ethylidene)hydrazine-1-carboximidamide. Light brown powder (175 mg, 72%). ¹H NMR (DMSO-d₆) δ: 7.75 (d, J=8.1 Hz, 2H), 7.25 (d, J=8.1 Hz, 2H), 5.94 (brs, 2H), 5.59 (brs, 2H), 2.96 (t, J=7.1 Hz, 2H), 2.20 (s, 3H), 1.61-1.50 (m, 2H), 1.40-1.24 (m, 10H), 0.87 (t, J=7.1 Hz, 3H); ¹³C NMR (DMSO-d₆); δ 160.04, 147.22, 137.93, 135.68, 128.07, 126.34, 32.59, 31.98, 29.07, 28.59, 28.23, 22.54, 14.40, 13.66; MS (m/z); 320.49 (M⁺, 100%).

2-(1-(4-(Nonylthio)phenyl)ethylidene)hydrazine-1-carboximidamide. Light brown powder (170 mg, 70%). ¹H NMR (DMSO-d₆) δ: 7.74 (d, J=8.1 Hz, 2H), 7.25 (d, J=8.1 Hz, 2H), 5.84 (brs, 2H), 5.45 (brs, 2H), 2.96 (t, J=7.1 Hz, 2H), 2.20 (s, 3H), 1.59-1.55 (m, 2H), 1.40-1.24 (m, 12H), 0.87 (t, J=7.1 Hz, 3H); ¹³C NMR (DMSO-d₆); δ 160.14, 147.11, 138.06, 135.54, 128.10, 126.30, 32.58, 31.73, 29.34, 29.07, 28.54, 22.55, 14.41, 13.62; MS (m/z); 334.52 (M⁺, 100%).

2-(1-(4-(Isobutylthio)phenyl)ethylidene)hydrazine-1-carboximidamide. Yellow solid (215 mg, 85%). ¹H NMR (DMSO-d₆) δ: 8.54 (brs, 2H), 8.41 (brs, 2H), 7.88 (d, J=8.1 Hz, 2H), 7.32 (d, J=8.1 Hz, 2H), 2.92 (d, J=7.2 Hz, 2H), 2.34 (s, 3H), 1.88-1.78 (m, 1H), 1.00 (d, J=7.4 Hz, 6H): ¹³C NMR (DMSO-d₆); δ 157.39, 150.78, 139.08, 134.87, 127.52, 127.44, 40.69, 28.18, 22.16, 14.60; MS (m/z); 264.39 (M⁺, 100%).

2-(1-(4-((Cyclobutylmethyl)thio)phenyl)ethylidene)hydrazine-1-carboximidamide. Yellow solid (165 mg, 66%). ¹H NMR (DMSO-d₆) δ: 7.77 (d, J=8.1 Hz, 2H), 7.26 (d, J=8.1 Hz, 2H), 5.70 (brs, 2H), 5.42 (brs, 2H), 3.09 (d, J=7.2 Hz, 2H), 2.98-2.53 (m, 1H), 2.21 (s, 3H), 2.01-1.26 (m, 6H); ¹³C NMR (DMSO-d₆); δ 160.11, 147.31, 137.77, 135.84, 128.68, 126.41, 39.01, 34.74, 31.91, 17.99, 13.72; MS (m/z); 276.40 (M⁺, 100%).

2-(1-(4-(Isopentylthio)phenyl)ethylidene)hydrazine-1-carboximidamide. Yellow solid (215 mg, 86%). ¹H NMR (DMSO-d₆) δ: 7.75 (d, J=8.1 Hz, 2H), 7.26 (d, J=8.1 Hz, 2H), 5.85 (brs, 2H), 5.46 (brs, 2H), 2.98 (t, J=7.2 Hz, 2H), 2.20 (s, 3H), 1.73-1.66 (m, 1H), 1.65-1.44 (m, 2H), 0.90 (d, J=7.4 Hz, 6H); ¹³C NMR (DMSO-d₆); δ 160.11, 147.13, 138.05, 135.53, 128.07, 126.33, 38.14, 30.67, 27.32, 22.60, 13.63; MS (m/z); 278.41 (M⁺, 100%).

2-(1-(4-((2-Ethylbutyl)thio)phenyl)ethylidene)hydrazine-1-carboximidamide. White powder (217 mg, 87%). ¹H NMR (DMSO-d₆) δ: 7.74 (d, J=8.1 Hz, 2H), 7.27 (d, J=8.1 Hz, 2H), 5.86 (brs, 2H), 5.48 (brs, 2H), 2.93 (d, J=4.2 Hz, 2H), 2.20 (s, 3H), 1.48-1.34 (m, 5H), 0.87 (d, J=7.4 Hz, 6H); ¹³C NMR (DMSO-d₆); δ 160.12, 147.11, 138.01, 136.00, 128.15, 126.30, 36.72, 31.91, 24.95, 13.63, 11.16; MS (m/z); 292.44 (M*, 100%).

2-(1-(4-(Cyclopentylthio)phenyl)ethylidene)hydrazine-1-carboximidamide. Biege powder (190 mg, 76%). ¹H NMR (DMSO-d₆) δ: 7.75 (d, J=8.1 Hz, 2H), 7.28 (d, J=8.1 Hz, 2H), 5.93 (brs, 2H), 5.56 (brs, 2H), 2.73-2.54 (m, 1H), 2.21 (s, 3H), 2.08-1.38 (m, 8H); ¹³C NMR (DMSO-d₆); δ 160.11, 147.31, 138.12, 136.02, 129.10, 126.32, 45.15, 33.46, 33.04, 24.69, 13.66; MS (m/z); 276.40 (M⁺, 100%).

2-(1-(4-(Cyclohexylthio)phenyl)ethylidene)hydrazine-1-carboximidamide. Off-white powder (195 mg, 78%). ¹H NMR (DMSO-d₆) δ: 7.76 (d, J=8.1 Hz, 2H), 7.33 (d, J=8.1 Hz, 2H), 5.90 (brs, 2H), 5.53 (brs, 2H), 3.26-3.21 (m, 1H), 2.21 (s, 3H), 1.93-1.21 (m, 10H); ¹³C NMR (DMSO-d₆); δ 160.13, 147.14, 138.86, 133.79, 130.90, 126.31, 45.65, 33.25, 33.04, 25.76, 13.65; MS (m/z); 290.42 (M⁺, 100%).

2-(1-(4-((Cyclohexylmethyl)thio)phenyl)ethylidene)hydrazine-1-carboximidamide. Brown powder (160 mg, 65%). ¹H NMR (DMSO-d₆) δ: 7.75 (d, J=8.1 Hz, 2H), 7.25 (d, J=8.1 Hz, 2H), 5.96 (brs, 2H), 5.61 (brs, 2H), 2.90 (d, J=7.1 Hz, 2H), 2.73-2.66 (m, 1H), 2.21 (s, 3H), 1.86-0.93 (m, 10H); ¹³C NMR (DMSO-d₆); δ 159.87, 147.40, 137.70, 136.29, 127.88, 126.39, 39.14, 32.68, 32.54, 26.43, 26.35, 13.67; MS (m/z); 304.45 (M⁺, 100%).

2-(1-(4-(Benzylthio)phenyl)ethylidene)hydrazine-1-carboximidamide. Light-brown powder (187 mg, 75%). ¹H NMR (DMSO-d₆) δ: 7.73 (d, J=8.1 Hz, 2H), 7.38 (d, J=8.1 Hz, 2H), 7.32-7.21 (m, 5H), 5.86 (brs, 2H), 5.47 (brs, 2H), 4.24 (s, 2H), 2.19 (s, 3H); ¹³C NMR (DMSO-d₆); δ 160.16, 147.05, 138.36, 138.11, 135.15, 129.25, 128.84, 128.46, 127.49, 126.23, 37.24, 13.61; MS (m/z); 298.40 (M⁺, 100%).

2-(1-(4-(Phenylethylthio)phenyl)ethylidene)hydrazine-1-carboximidamide. Brown powder (220 mg, 90%). ¹H NMR (DMSO-d₆) δ: 7.79 (d, J=8.1 Hz, 2H), 7.32-7.20 (m, 7H), 5.86 (brs, 2H), 5.47 (brs, 2H), 3.26 (t, J=7.2 Hz, 2H), 2.91 (t, J=7.2 Hz, 2H), 2.21 (s, 3H); ¹³C NMR (DMSO-d₆); δ 160.16, 147.11, 140.49, 138.21, 135.16, 129.01, 128.83, 128.22, 126.75, 126.41, 35.19, 34.01, 13.65; MS (m/z); 312.43 (M⁺, 100%).

2-(1-(4-(Hexylthio)phenyl)ethylidene)hydrazine-1-carboximidamide. Yellow solid: 7.7 (d, J=8.4 Hz, 2H), 7.3 (d, J=8.4 Hz, 2H), 5.7 (brs, 3H), 5.4 (brs, 3H), 2.8 (t, J=4.8 Hz, 2H), 2.1 (s, 3H), 1.5 (m, 2H), 1.2 (m, 8H), 0.7 (t, J=4.8 Hz, 3H): 13C NMR (DMSO-d6); δ 160.14, 147.14, 138.05, 135.57, 128.08, 126.31, 32.60, 31.25, 29.05, 28.25, 22.47, 14.34, 13.64; MS (m/z); 292.44 (M⁺, 100%).

The minimum inhibitory concentration (MIC) of tested compounds and control antibiotics was determined using the broth microdilution method according to the guidelines outlined by the Clinical and Laboratory Standards Institute (CLSI, Methods for Dilution Antimicrobial Susceptibility Tests for Bacteria That Grow Aerobically; Approved Standard. January 2012; Vol. Ninth Edition M07-A9. 32 No. 2.). Bacterial strains were grown aerobically overnight on tryptone soy agar plates at 37° C. Afterwards, a bacterial solution equivalent to 0.5 McFarland standard was prepared and diluted in cation-adjusted Mueller-Hinton broth (CAMHB) to achieve a bacterial concentration of about 5×10⁵ CFU/mL and seeded in 96-well plates. Compounds and control drugs were added in the first row of the 96-well plates and serially diluted along the plates. Plates were then, incubated aerobically at 37° C. for 18-20 hours. MICs reported here are the minimum concentration of the compounds and control drugs that completely inhibited the visual growth of bacteria.

Multi-step resistance testing was conducted as reported earlier (PLOS One, 12 (2017) e0182821). A broth microdilution assay was used to determine the MIC for the MRSA strain USA300 after being exposed for 14 consecutive passages to either the example compound of formula 75, or rifampicin, as a control antibiotic. Briefly, different concentrations of TBT4 and rifampicin were prepared in 10 mL Mueller-Hinton (MH) broth (0.125 to 32 μg/ml and 0.003 to 1024 μg/mL, respectively) and they were inoculated with 10 μL of an overnight culture of S. aureus strain USA300 and then incubated at 37° C., with shaking at 180 rpm, for 24 h. The highest example compound of formula 75 and rifampicin concentrations that showed positive growth were used to inoculate another set of MH broth with increasing concentrations as mentioned above. Resistance was classified as a greater than four-fold increase in the initial MIC (Antimicrob. Agents Chemother., 55 (2011) 1177-1181).

The present invention has been described and illustrated with reference to an exemplary embodiment; however, it will be understood by those skilled in the art that various changes may be made, and equivalents may be substituted for elements thereof without departing from the scope of the invention as set out in the following claims. Therefore, it is intended that the invention is not limited to the embodiments disclosed herein.

What is claimed is:

1. A compound selected from the group consisting of formula Iₐ, I_{b}, and I_{c}, 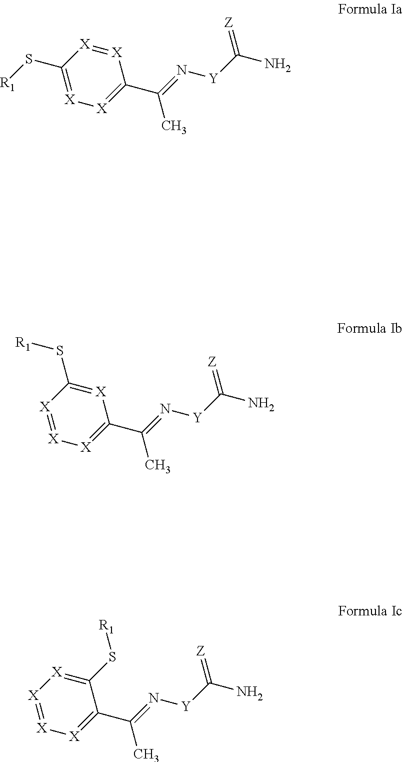
wherein: R₁ is selected from the group consisting of: a linear or branched akyl chain, unsubstituted or substituted with one or more halides, CN, OH, NO₂, NH, NH₂; an alkenyl, cycloalkyl, alkynyl or cycloakenyl group; and a cycloalkyl or cycloakenyl group connected with a S atom, via a straight or branched one to nine C chain;
each X is independently selected from the group consisting of: C, CH, N, O, S, a halogen, a halogenated C, alkyl, CN, OH, NO₂, NH, NH₂, and a substituted or unsubstituted three to eight C ring;
Y is selected from the group consisting of: CH₂, NH, N-alkyl, and a substituted or unsubstituted three to eight C ring; and
Z is selected from the group consisting of: O, S, NH, N-alkyl, OH, and a substituted or unsubstituted three to eight C ring.

2. The compound of claim 1, wherein R₁ is a side group selected from the group consisting of formulas (a)-(o): 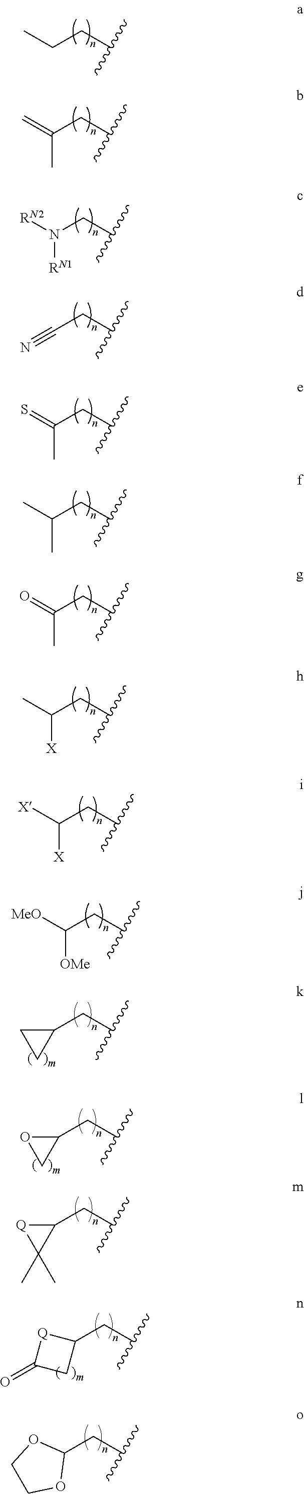
wherein the value of n is between 0 and 12 and the value of m is between 0 and 7;
wherein Q is selected from the group consisting of: NH, O, S, and N—R^{N1};
wherein R^{N1} and R^{N2} are, independently, alkyl or cycloalkyl groups with between 1 and 7 C atoms, unsubstituted or substituted with one or more halogen atoms; and
wherein X and X′ are, independently, selected from the group consisting of: F, Cl, Br, and I.

3. The compound of claim 2, wherein the compound is a compound of formula 1.

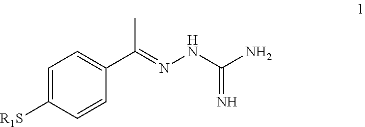

4. The compound of claim 2, wherein the compound is a compound of formula 2.

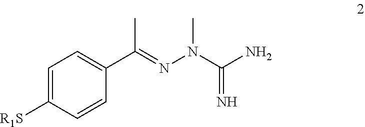

5. The compound of claim 2, wherein the compound is a compound of formula 3.

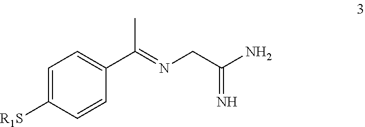

6. The compound of claim 2, wherein the compound is a compound of formula 4.

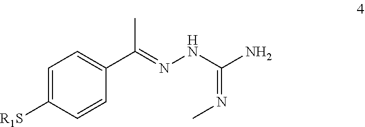

7. The compound of claim 2, wherein the compound is a compound of formula 5.

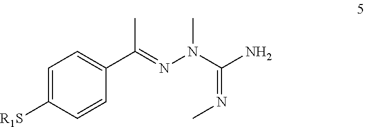

8. The compound of claim 2, wherein the compound is a compound of formula 6.

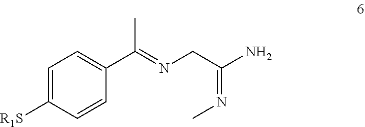

9. The compound of claim 2, wherein the compound is a compound of formula 7.

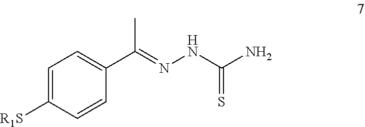

10. The compound of claim 2, wherein the compound is a compound of formula 8.

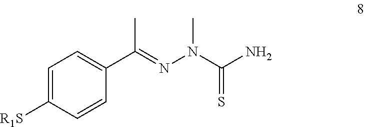

11. The compound of claim 2, wherein the compound is a compound of formula 9.

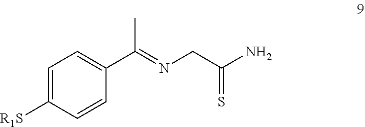

12. The compound of claim 2, wherein the compound is a compound of formula 10.

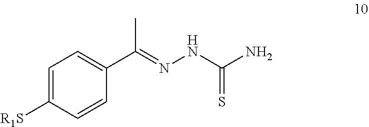

13. The compound of claim 2, wherein the compound is a compound of formula 11.

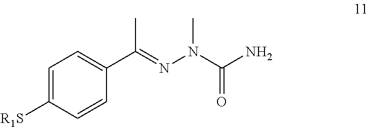

14. The compound of claim 2, wherein the compound is a compound of formula 12.

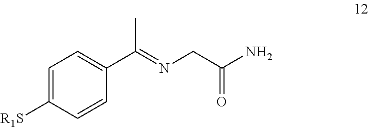

15. The compound of claim 2, wherein the compound is a compound of formula 13.

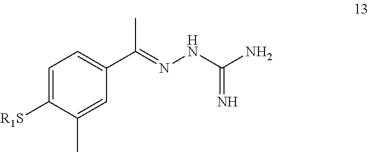

16. The compound of claim 2, wherein the compound is a compound of formula 14.

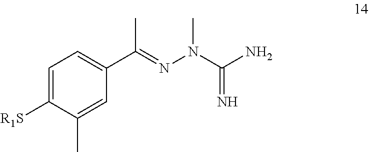

17. The compound of claim 2, wherein the compound is a compound of formula 15.

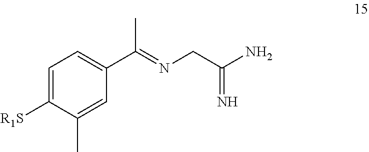

18. The compound of claim 2, wherein the compound is a compound of formula 16.

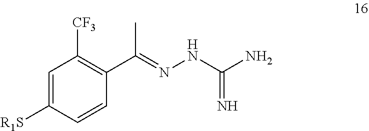

19. The compound of claim 2, wherein the compound is a compound of formula 17.

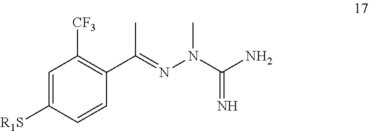

20. The compound of claim 2, wherein the compound is a compound of formula 18.

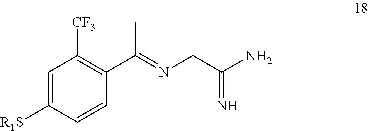

21. The compound of claim 2, wherein the compound is a compound of formula 19.

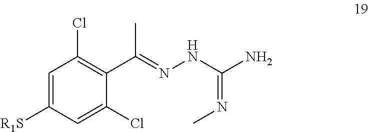

22. The compound of claim 2, wherein the compound is a compound of formula 20.

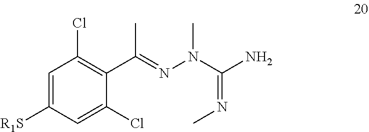

23. The compound of claim 2, wherein the compound is a compound of formula 21.

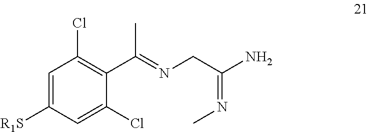

24. The compound of claim 2, wherein the compound is a compound of formula 22.

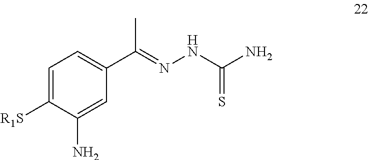

25. The compound of claim 2, wherein the compound is a compound of formula 23.

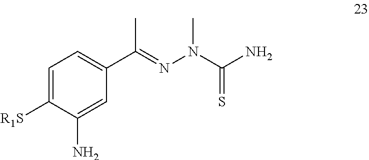

26. The compound of claim 2, wherein the compound is a compound of formula 24.

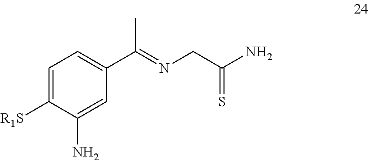

27. The compound of claim 2, wherein the compound is a compound of formula 25.

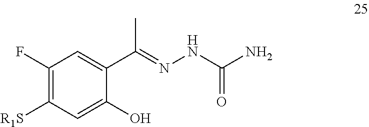

28. The compound of claim 2, wherein the compound is a compound of formula 26.

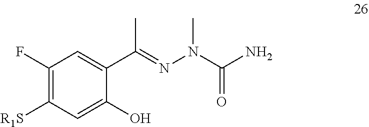

29. The compound of claim 2, wherein the compound is a compound of formula 27.

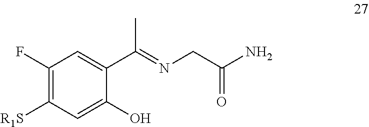

30. The compound of claim 2, wherein the compound is a compound of formula 28.

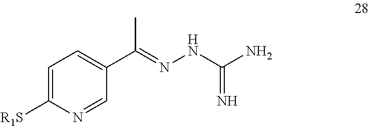

31. The compound of claim 2, wherein the compound is a compound of formula 29.

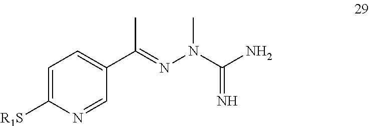

32. The compound of claim 2, wherein the compound is a compound of formula 30.

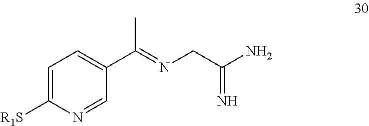

33. The compound of claim 2, wherein the compound is a compound of formula 31.

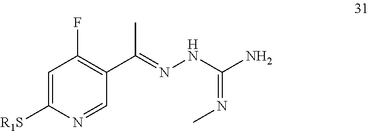

34. The compound of claim 2, wherein the compound is a compound of formula 32.

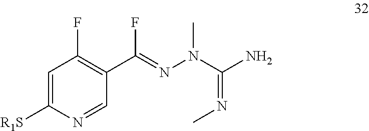

35. The compound of claim 2, wherein the compound is a compound of formula 33.

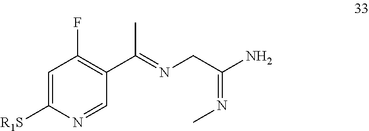

36. The compound of claim 2, wherein the compound is a compound of formula 34.

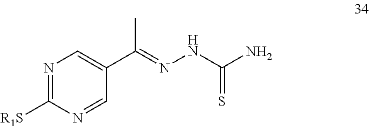

37. The compound of claim 2, wherein the compound is a compound of formula 35.

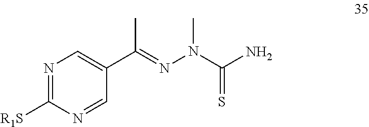

38. The compound of claim 2, wherein the compound is a compound of formula 36.

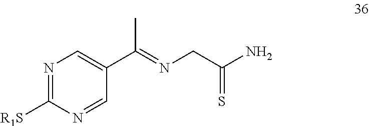

39. The compound of claim 2, wherein the compound is a compound of formula 37.

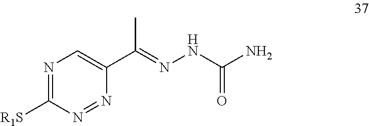

40. The compound of claim 2, wherein the compound is a compound of formula 38.

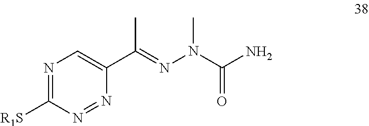

41. The compound of claim 2, wherein the compound is a compound of formula 39.

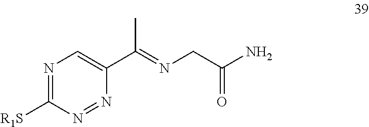

42. A compound selected from the group consisting of formula IIₐ, II_{b}, and II_{c}, 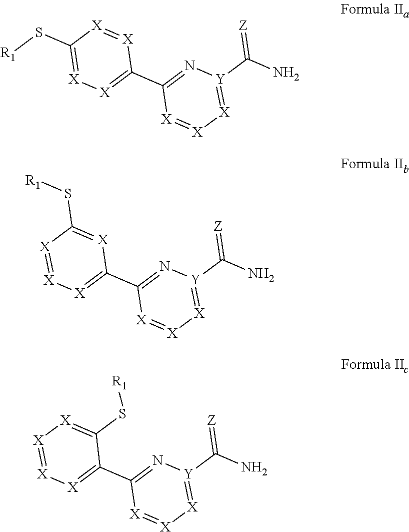
wherein: R₁ is selected from the group consisting of: a linear or branched akyl chain, unsubstituted or substituted with one or more halides, CN, OH, NO₂, NH, NH₂; an alkenyl, cycloalkyl, alkynyl or cycloakenyl group; and a cycloalkyl or cycloakenyl group connected with a S atom, via a straight or branched one to nine C chain;
each X is independently selected from the group consisting of: C, CH, N, O, S, a halogen, a halogenated C, alkyl, CN, OH, NO₂, NH, NH₂, and a substituted or unsubstituted three to eight C ring;
Y is selected from the group consisting of: CH₂, NH, N-alkyl, and a substituted or unsubstituted three to eight C ring; and
Z is selected from the group consisting of: O, S, NH, N-alkyl, OH, and a substituted or unsubstituted three to eight C ring.

43. The compound of claim 42, wherein R₁ is a side group selected from the group consisting of formulas (a)-(o). 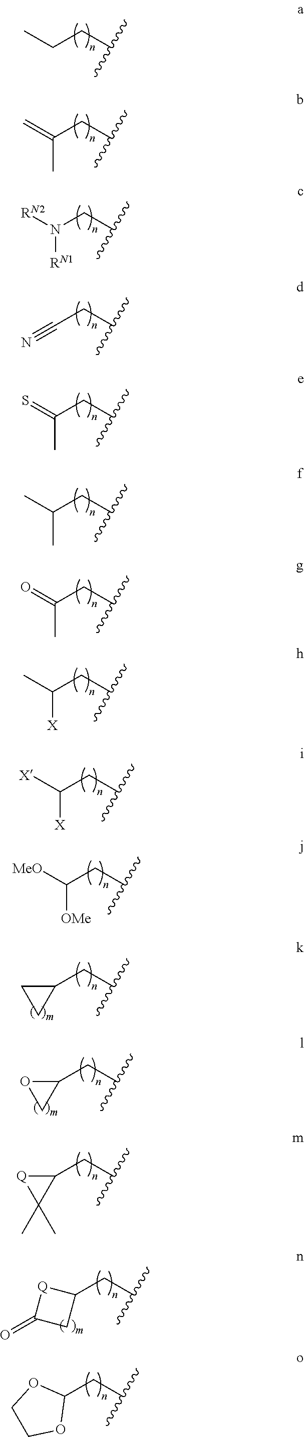
wherein the value of n is between 0 and 12 and the value of m is between 0 and 7;
wherein Q is selected from the group consisting of: NH, O, S, and N—R^{N1};
wherein R^{N1} and R^{N2} are, independently, alkyl or cycloalkyl groups with between 1 and 7 C atoms, unsubstituted or substituted with one or more halogen atoms; and
wherein X and X′ are, independently, selected from the group consisting of: F, Cl, Br, and I.

44. The compound of claim 43, wherein the compound is a compound of formula 40.

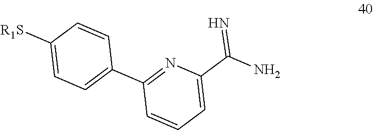

45. The compound of claim 43, wherein the compound is a compound of formula 41.

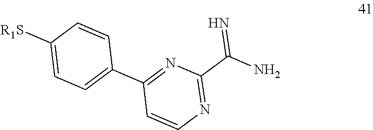

46. The compound of claim 43, wherein the compound is a compound of formula 42.

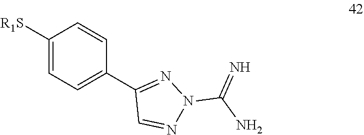

47. A compound selected from the group consisting of formula IIIₐ, III_{b}, and III_{c}, 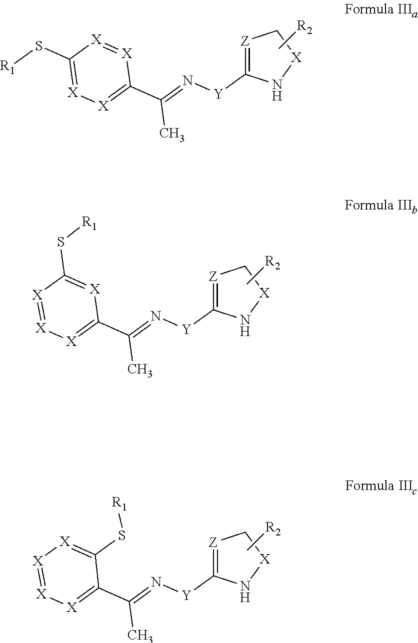
wherein: R₁ is selected from the group consisting of: a linear or branched akyl chain, unsubstituted or substituted with one or more halides, CN, OH, NO₂, NH, NH₂; an alkenyl, cycloalkyl, alkynyl or cycloakenyl group; and a cycloalkyl or cycloakenyl group connected with a S atom, via a straight or branched one to nine C chain;
R₂ is selected from the group consisting of: H, a straight or branched one to four C chain, unsubstituted or substituted with one or more halides, CN, OH, NO₂, NH, NH₂.
each X is independently selected from the group consisting of: C, CH, N, O, S, a halogen, a halogenated C, alkyl, CN, OH, NO₂, NH, NH₂, and a substituted or unsubstituted three to eight C ring;
Y is selected from the group consisting of: CH₂, NH, N-alkyl, and a substituted or unsubstituted three to eight C ring; and
Z is selected from the group consisting of: O, S, NH, N-alkyl, OH, and a substituted or unsubstituted three to eight C ring.

48. The compound of claim 47, wherein R₁ is a side group selected from the group consisting of formulas (a)-(o). 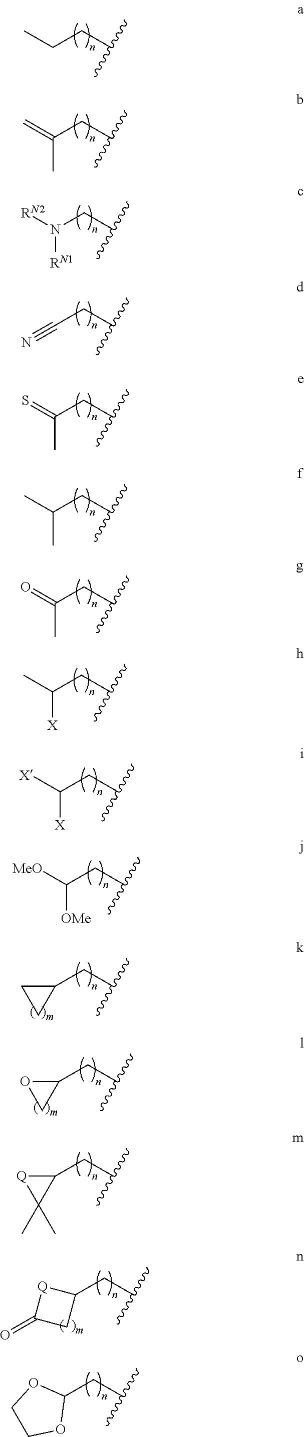
wherein the value of n is between 0 and 12 and the value of m is between 0 and 7;
wherein Q is selected from the group consisting of: NH, O, S, and N—R^{N1},
wherein R^{N1} and R^{N2} are, independently, alkyl or cycloalkyl groups with between 1 and 7 C atoms, unsubstituted or substituted with one or more halogen atoms; and
wherein X and X′ are, independently, selected from the group consisting of: F, Cl, Br, and I.

49. The compound of claim 48, wherein the compound is a compound of formula 43.

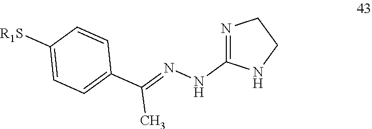

50. The compound of claim 48, wherein the compound is a compound of formula 44.

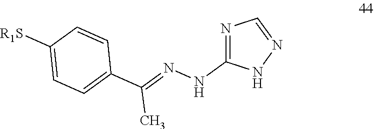

51. The compound of claim 48, wherein the compound is a compound of formula 45.

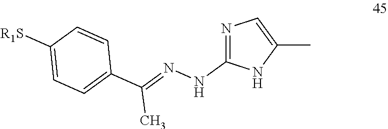

52. A compound selected from the group consisting of formula IVₐ, IV_{b}, IV_{c}, IV_{d}, IVₑ, and IV_{f}, 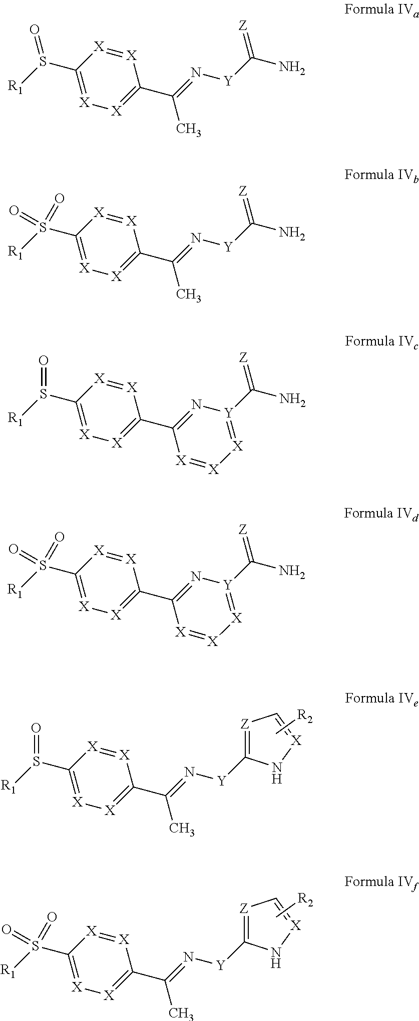
wherein: R₁ is selected from the group consisting of: a linear or branched akyl chain, unsubstituted or mono-, di-, or multi-substituted with a halide, CN, OH, NO₂, NH, NH₂; an alkenyl, cycloalkyl, alkynyl or cycloakenyl group; and a cycloalkyl or cycloakenyl group connected with a S atom, via a straight or branched one to nine C chain;
R₂ is selected from the group consisting of: H, a straight or branched one to four C chain, unsubstituted or mono-, di-, or multi-substituted with a halide, CN, OH, NO₂, NH, NH₂.
each X is independently selected from the group consisting of: C, CH, N, O, S, a halogen, a halogenated C, alkyl, CN, OH, NO₂, NH, NH₂, and a substituted or unsubstituted three to eight C ring;
Y is selected from the group consisting of: CH₂, NH, N-alkyl, and a substituted or unsubstituted three to eight C ring; and
Z is selected from the group consisting of: O, S, NH, N-alkyl, OH, and a substituted or unsubstituted three to eight C ring.

53. The compound of claim 52, wherein R₁ is a side group selected from the group consisting of formulas (a)-(o).

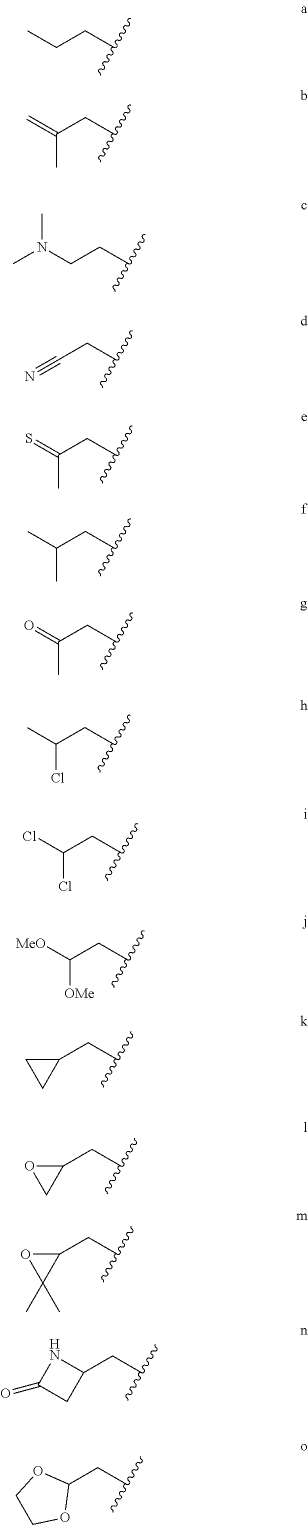

54. The compound of claim 53, wherein the compound is a compound of formula 46.

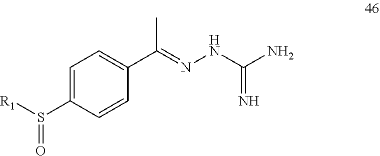

55. The compound of claim 53, wherein the compound is a compound of formula 47.

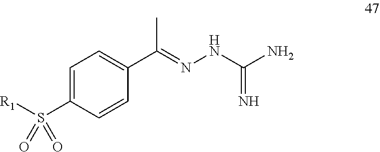

56. The compound of claim 53, wherein the compound is a compound of formula 48.

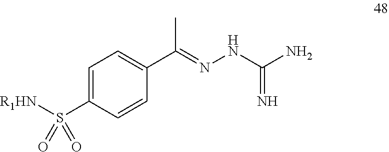

57. The compound of claim 53, wherein the compound is a compound of formula 49.

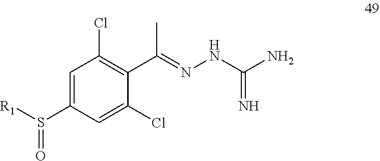

58. The compound of claim 53, wherein the compound is a compound of formula 50.

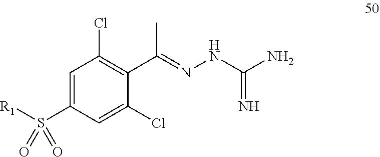

59. The compound of claim 53, wherein the compound is a compound of formula 51.

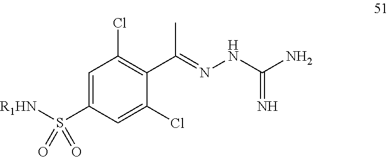

60. The compound of claim 53, wherein the compound is a compound of formula 52.

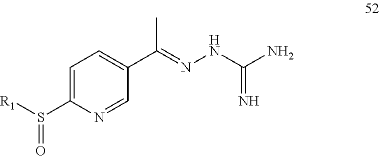

61. The compound of claim 53, wherein the compound is a compound of formula 53.

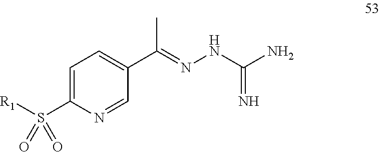

62. The compound of claim 53, wherein the compound is a compound of formula 54.

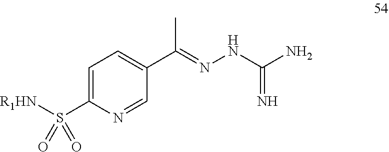

63. The compound of claim 53, wherein the compound is a compound of formula 55.

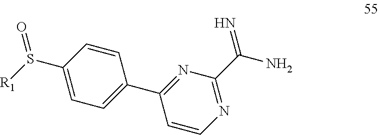

64. The compound of claim 53, wherein the compound is a compound of formula 56.

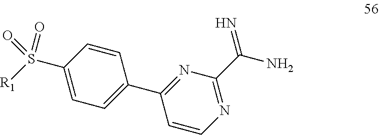

65. The compound of claim 53, wherein the compound is a compound of formula 57.

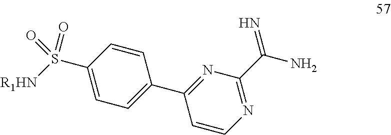

66. The compound of claim 53, wherein the compound is a compound of formula 58.

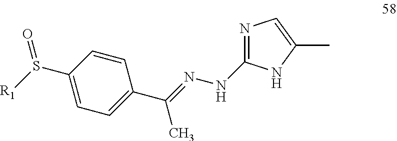

67. The compound of claim 53, wherein the compound is a compound of formula 59.

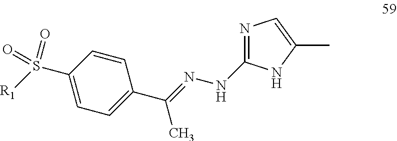

68. The compound of claim 53, wherein the compound is a compound of formula 60.

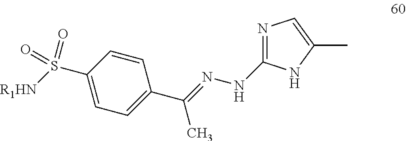

69. The compound of any of claim 2, 43, 48, or 53, wherein the compound in in the form of a salt with one or more antimicrobial compounds.

70. The compound of claim 69, wherein at least one of the one or more antimicrobial compounds is selected from the group consisting of penicillins, penams, carbapenams, clavams, carbacephems, oxacephems, monobactam, quinolones, and fluoroquinolones.

71. The compound of claim 70, wherein the compound is a compound of formula 61.

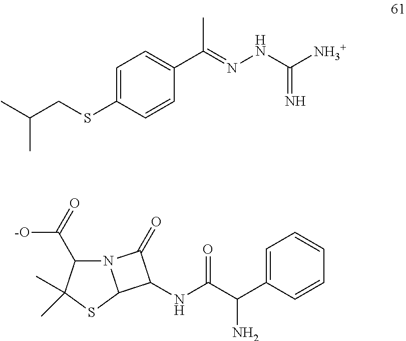

72. The compound of claim 70, wherein the compound is a compound of formula 62.

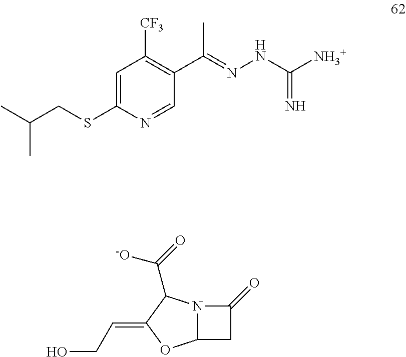

73. The compound of claim 70, wherein the compound is a compound of formula 63.

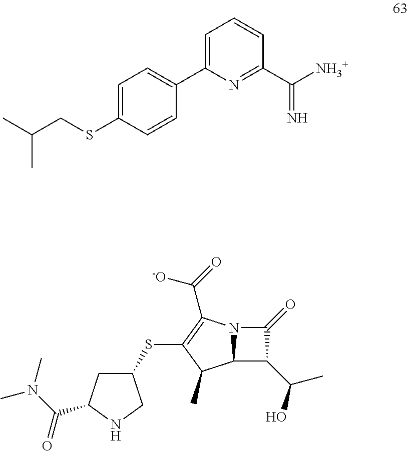

74. The compound of claim 70, wherein the compound is a compound of formula 64.

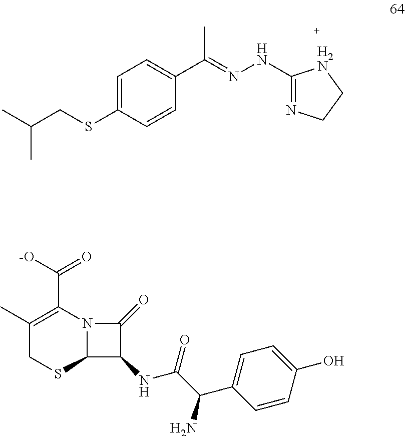

75. The compound of claim 70, wherein the compound is a compound of formula 65.

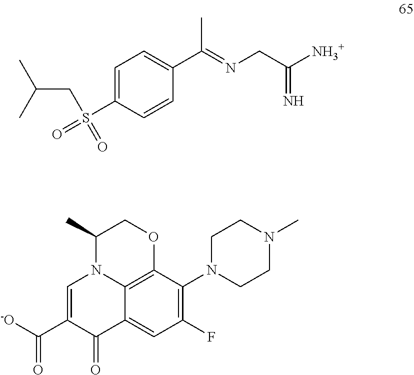

76. The compound of claim 70, wherein one of the one or more antimicrobial compounds is a β-lactamase inhibitor.

77. The compound of claim 76, wherein the compound is a compound of formula 66.

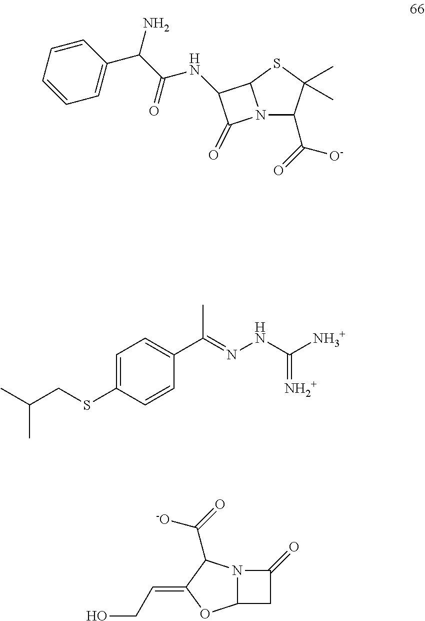

78. The compound of claim 76, wherein the compound is a compound of formula 67.

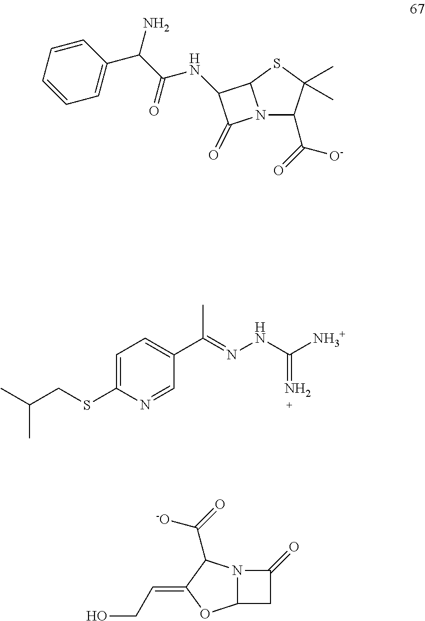

79. The compound of any of claim 1, 42, 47, or 52, wherein the R₁ side chain comprises one or more ketonic, aldehydic, or epoxy groups.

80. The compound of claim 79, wherein one or more of the X groups are a substituted or unsubstituted five or six C ring.

81. The compound of claim 79, wherein Y is a substituted or unsubstituted five or six C ring.

82. The compound of claim 81, wherein Y is a substituted or unsubstituted imidazoline, imidazole, triazole, pyridine, or pyrimidine.

83. The compound of claim 1, wherein each X is C and both Y and Z are NH.

84. The compound of claim 83, wherein the compound is a compound of formula 72.

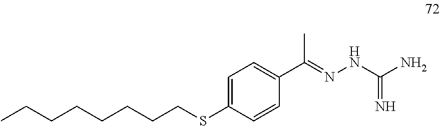 [SMILES: CCCCCCCCSc1ccc(/C(C)=N/NC(=N)N)cc1]

85. The compound of claim 83, wherein the compound is a compound of formula 74.

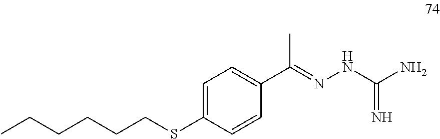 [SMILES: CCCCCCSc1ccc(/C(C)=N/NC(=N)N)cc1]

86. The compound of claim 83, wherein the compound is a compound of formula 75.

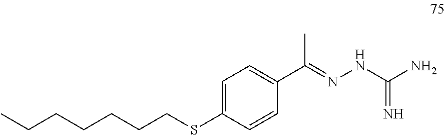 [SMILES: CCCCCCCSc1ccc(/C(C)=N/NC(=N)N)cc1]

87. A pharmaceutical composition, comprising a compound of any of claims 1-86 or a pharmaceutically acceptable salt thereof and one or more pharmaceutically acceptable excipients.

88. The pharmaceutical composition of claim 83, comprising a second therapeutically active compound.

89. The pharmaceutical composition of claim 83, wherein the pharmaceutical composition is in the form of a topical application.

90. A method of treating a patient with a bacterial infection, comprising administering a therapeutically effective amount of a compound of any of claims 1-86 to a patient in need thereof.

91. The method of claim 90, wherein the bacterial infection is a polymicrobial skin infection and the compound is administered in the form of a topical application.
